(19) United States
(12) Patent Application Publication
(10) Pub. No.: US 2014/0256581 A1
(43) Pub. Date: Sep. 11, 2014

(54) VIRAL NANOARRYS AND SENSORS COMPRISING THE SAME

(71) Applicants: Hadar Ben-Yoav, Rockville, MD (US); Reza Ghodssi, Potomac, MD (US)

(72) Inventors: Hadar Ben-Yoav, Rockville, MD (US); Reza Ghodssi, Potomac, MD (US)

(21) Appl. No.: 14/190,060

(22) Filed: Feb. 25, 2014

Related U.S. Application Data
(60) Provisional application No. 61/768,808, filed on Feb. 25, 2013.

(51) Int. Cl. G01N 33/554; C12Q 1/68; C12N 15/10

(52) U.S. Cl. CPC G01N 33/554; C12N 15/1037; C12Q 1/6837
USPC 506/9; 506/18; 506/14; 506/16

(57) ABSTRACT

The present invention provides arrays comprising polypeptides associated with viruses that are immobilized to an electrode in a directional orientation such that the surface area of the electrode and the sensitivity of the electrode is increased. The invention also provides for methods of diagnosing and/or prognosing a certain disease or disorder through contacting a sample from a patient with an array comprising the polypeptides associated with viruses.

### CROSS-REFERENCE TO RELATED APPLICATIONS

This application is a non-provisional application filed under 35 U.S.C. §120, which claims priority to U.S. Provisional Ser. No. 61/768,808, filed on Feb. 25, 2013, which is herein incorporated by reference in its entirety.

### STATEMENT REGARDING FEDERALLY SPONSORED RESEARCH

This invention was made with government support under grant number CMMI0927693 awarded by the National Science Foundation and under grant number W911NF11101 award by the United States Army. The United States government has certain rights in this invention.

### FIELD OF THE INVENTION

The invention relates generally to devices that detect or isolate molecules or substances based upon their affinity to polypeptides and/or metal coatings immobilized to a surface of the device. In some embodiments, the polypeptides include whole viruses or viral envelope proteins. The invention also relates to methods of making the device and methods of diagnosing patients with particular disorders based upon the presence or absence or quantity of a substance or molecule on the device.

### BACKGROUND OF THE INVENTION

There are a wide variety of contexts in biology and medicine when it is important or useful to be able to detect the presence, absence, or quantity of cells, pathogens, molecules (including nucleic acid molecules and protein) in a solution, to separate the aforementioned substances from a solution or surface and to distinguish different types of substances from one another, and/or to identify, characterize, or define particular substances in a water sample, bodily fluid sample or a food sample. Current methods of isolation, differentiation, and characterization can be improved. Identification and characterization of certain cellular properties or expression profiles can be used for diagnosis or prognosis for certain disease states. Identification and characterization of certain pathogens, molecules, or nucleic acids can be used for food safety and reduction of environmental hazard. One of the challenges with sensmg devices is the low signal-to-noise ratio that decreases the performance of sensors. By increasing the signal, electrochemical sensors can improve their detection efficiency resulting in more sensitive sensors [1].

### SUMMARY OF INVENTION

The present invention relates to devices and kits for, and methods of detection of, the presence or absence and/or quantification of analytes, pathogens, substances or molecules by viruses or recombinant viral proteins immobilized on a solid surface. In some embodiments, the invention relates to detecting the presence, absence, or quantity of an analyte or substance in solution or on a surface. In some embodiments, the invention relates to detecting the presence, absence, or quantity of a molecule in solution or on a surface. In some embodiments, the invention relates to a method of detecting the presence, absence, or quantity of a pathogen or substance derived from a pathogen in solution or on a surface. In some embodiments, the device comprises immobilized virus or viral components directionally oriented on a surface. In some embodiments, the device comprises a plurality of immobilized virus or viral proteins. In some embodiments, the virus is a Tobacco Mosaic Virus (TMV) or a TMV viral protein or modified analog thereof. In some embodiments, the device is a sensor comprising at least three surfaces arranged in at least three successive layers, wherein the first surface comprises a metal surface area, the second surface comprises one or a plurality of viruses or viral proteins, wherein, optionally, at least a portion of the one or plurality of viruses or viral proteins or analogs thereof are directionally immobilized to the first surface; and wherein the third surface comprises at least one metal coating in contact with the at least one or plurality of viruses, viral proteins or analogs thereof on the second surface. In some embodiments, at least one portion of the third surface comprises two layers of metal ions. In some embodiments, at least one portion of the third surface comprises one layer of nickel and one layer of gold. In some embodiments, the invention relates to a sensor or device comprising at least three surfaces arranged, at least partially, in at least three successive layers, wherein the first surface comprises a metal electroconductive surface area, the second surface comprises one or a plurality of viruses, wherein, optionally, at least a portion of the one or plurality of viruses or viral proteins are optionally directionally immobilized to the first surface on a vertical axis; wherein the third surface comprises at least one metal coating in contact with a portion of the surface area of the at least one or plurality of viruses or viral proteins on the second surface. In some embodiments, at least one of the at least three surfaces are arranged in a planar or substantially planar orientation.

In some embodiments, the at least one portion of the third surface comprises two layers of metal. In some embodiments, the third surface comprises at least a first area of nickel and at least a second area of gold immediately adjacent to the nickel.

The invention also relates to methods of detecting the presence, absence, or quantity of a molecule, analyte, or pathogen in a solution or on a surface, the method comprising exposing any device or sensor disclosed herein to the solution or the surface. In some embodiments, the method comprises contacting a swab or element to a test surface or test solution comprising a target molecule or suspected of comprising a target molecule, exposing the swab or element to any device or sensor disclosed herein. The invention further relates to the method comprises contacting a swab or element to a testing surface comprising a target molecule or suspected of comprising a target molecule, storing the swab or element for a period of time in a test solution, and exposing the swab or element to any device or sensor disclosed herein to detect the presence, absence, or quantity of the target molecule.

The invention further relates to a method of isolating a molecule or substance comprising exposing any device or sensor disclosed herein to a solution or a surface comprising the molecule or substance or to a solution or surface suspected of comprising the molecule or substance. In some embodiments, the target molecule is a pathogen, radioactive isotope, or heavy metal. In some embodiments, the method comprises contacting a swab or element to a testing surface or test solution comprising the molecule or substance or suspected of comprising the molecule, exposing the swab or element to any device or sensor disclosed herein. In some embodiments, the method of isolating a molecule comprises contacting a swab or element to a testing surface comprising a molecule or substance or suspected of comprising the molecule or the substance, storing the swab or element for a period of time optionally in a test solution, and exposing the swab or element to any device or sensor disclosed herein to detect the presence, absence, or quantity of the molecule or substance.

The invention also relates to a device or sensor comprising a solid surface comprising at least one or a plurality viruses or viral proteins oriented in a longitudinal fashion. In some embodiments, the solid surface is an electrode operably linked to one or a plurality of ammeters and/or voltmeters.

The invention relates to any of the devices or sensors disclosed herein manufactured by directional deposition of at least one or a plurality of viruses or viral proteins to a solid surface, depositing a first metal coating to the at least one or a plurality of viruses or viral proteins with a metal, and optionally depositing a second metal coating over the at least one or plurality of viruses or viral proteins. In some embodiments, invention relates to any of the devices or sensors disclosed herein manufactured by directional deposition of at least one or a plurality of viruses or viral proteins to a solid surface, depositing a first metal coating to the at least one or a plurality of viruses or viral proteins with a metal, and optionally depositing a second metal coating over the at least one or plurality of viruses or viral proteins; and covalently bonding a molecule, linker, linker and molecule or probe to the one ore more viruses or viral proteins or the first or second metal surface. In some embodiments, the molecule bound to the linker or metal surface or one or plurality of viruses or viral proteins is a nucleic acid molecule, antibody, or probe.

The invention further relates to any of the devices or sensors disclosed herein wherein the at least one virus or viral protein comprises TMV or a protein derived from TMV covalently bound to probe by at least one linker or chemical group. In some embodiments, the probe is a single stranded nucleic acid, double-stranded nucleic acid, or an analog thereof.

The invention further relates to a device or sensor, or a kit comprising any of the devices or sensors or arrays disclosed herein, comprising at least a one or a plurality of viruses or viral proteins vertically oriented on a solid surface and capable of electrochemical detection of one or more analytes, one or more molecules, one or more substances, and/or one or more pathogens in a solution or on a surface. In some embodiments the virus, surface of one or a plurality of viruses, or a probe covalently bound to the one or plurality of metal surfaces possesses an affinity for one or more target molecules or substances and is sufficient to distinguish or characterize a surface or solution comprising one or more target molecules as compared with at least one other reference surface. For example, in some embodiments, the electrochemical detection is sufficient to distinguish or characterize the presence, absence, or quantity of cells, pathogens, substances and/or molecules from a particular cell type or virus (e.g., eukaryotic cell, prokaryotic cell, virus, etc) from cells of one or more other types. In some embodiments, the device, sensor, or kits comprising the same and disclosed herein allows isolation of a particular pathogen or molecule or substance from a solution or surface.

In accordance with the present invention, the invention further relates to a method of diagnosing a condition, disorder, or disease in a subject comprising exposing a bodily fluid or test sample from the subject to any of the devices, sensors, or compositions disclosed herein, and detecting the presence, absence, or quantity of a target molecule or substance. In some embodiments the disorder is a psychotic disorder. In some embodiments, the method of diagnosing further comprises comparing the signal or quantity of the target molecule or substance in the test sample relative to a signal or quantity of the target molecule or substance in a subject known not to be afflicted by the condition, disorder, or disease, and diagnosing the subject as having the condition, disorder, or disease if the absence, presence or quantity of test sample or substance is distinguishable from absence, presence or quantity of the substance or molecule from the test sample of a subject known not to be affected by the condition, disorder, or disease. In some embodiments, the step of detecting the presence, absence or quantity of a test molecule or substance in a test sample is performed in series or in parallel to the step of detecting the presence, absence, or quantity of the target molecule or substance in a control sample which is optionally taken from a subject known not to be afflicted by the condition, disorder, or disease in the case of diagnosis or prognosis of a condition.

Further in accordance with the present invention, the device and/or sensors and/or arrays disclosed herein are used to identify and/or characterize the safety of food or water. For example, in some embodiments the devices or sensors provided herein are used to identify food samples or water samples comprising a pathogen from food samples or water samples that do not comprises a pathogen.

In some embodiments, the device and/or sensors and/or arrays disclosed herein are used to identify and/or characterize a patient as being susceptible to a particular disease, disorder, or condition due to the presence or quantity of nucleic acid expression or protein expression as compared to the presence or quantity of nucleic acid expression or protein expression in a sample from a patient that is not susceptible to the disease. In further embodiments, association values are used to identify and/or characterize the genetic background of a subject. In some embodiments, the device, sensor, or composition is used to measure association values of a sample from a subject and compare the association value to the association value of a sample from a control subject.

The invention provides an array of viruses or viral polypeptides, the array comprising: a solid support and a plurality of viruses vertically oriented along a longitudinal axis, wherein the one or plurality of viruses are immobilized to the solid support and wherein the solid support comprises a surface area capable of electroconductivity. In some embodiments, the solid support comprises at least partially metal substance optionally coated with a polymer in operable connection to one or a plurality of voltmeters or ammeters. In some embodiments, the solid support is coated with a metal an a polymer. In some embodiments, the polymer is polyacrylamide. In some embodiments, the solid support comprises a material chosen from: polystyrene (TCPS), glass, quarts, quartz glass, poly(ethylene terephthalate) (PET), polyethylene, polyvinyl difluoride (PVDF), polydimethylsiloxane (PDMS), polytetrafluoroethylene (PTFE), polymethylmethacrylate (PMMA), polycarbonate, polyolefin, ethylene vinyl acetate, polypropylene, polysulfone, polytetrafluoroethylene, silicones, poly(meth)acrylic acid, polyamides, polyvinyl chloride, polyvinylphenol, and copolymers and mixtures thereof.

The invention further relates to an array of polypeptides, the array comprising: a solid support and a plurality of viruses and/or viral proteins and/or analogs thereof, wherein the viruses and/or viral proteins and/or analogs thereof are immobilized to the solid support at least one addressable location of the array; and wherein the viral protein and/or analogs thereof two or more are chosen from TMV capsid sequence here or analogs thereof comprising at least 70%, 75%, 80%, 85%, 90%, 95%, 96%, 97%, 98%, or 99% sequence identity to TMV capsid sequence or any of the SEQ ID NOs described herein.

In some embodiments, the viruses, viral polypeptides, and/or analogs thereof are derived from a protein sequence of the Tobamovirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to a capsid protein of a virus in the Tobamovirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to SEQ ID NO:1: MPYTINSPSQ FVYLSSAYAD PVQLINLCTN ALGNQFQTQQ ARTTVQQQFA DAWKPVPSMT VRFPASDFYV YRYNSTLDPL ITALLNSFDT RNRIIEVDNQ PAPNTTEIVN ATQRVDDATV AIRASINNLA NELVRGTGMF NQAGFETASG LVWTTTPAT.

In some embodiments, the viruses, viral polypeptides, and/or analogs thereof are derived from a protein sequence of the Furovirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to a capsid protein of a virus in the Furovirus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to SEQ ID NO:2: MAVKSGYTVF NKELNVMANT HAYIRLSALL SQVETWQSTR TSVLSHLGIM LNGVSKLGER NFFSRSKRFG THTFDGDEIF CDLGGEGVSQ VLTRLIVALG AAKGEGAQSR NAKRGAPPAA GQIETEEQGQ TDQSLAISNA LGELMTYVSS KEYTMNECYT QDSFEAKYNL KWEGSS.

In some embodiments, the viruses, viral polypeptides, and/or analogs thereof are derived from a protein sequence of the Hordeivirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to a capsid protein of a virus in the Hordeivirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to SEQ ID NO:3: MPNVSLTAKG GGHYIEDQWD TQVVEAGVFD DWWVHVEAWN KFLDNLRGIN FSVASSRSQV AEYLAALDRD LPADVDRRFA GARGQIGSPN YLPAPKFFRL DKRTIAELTR LSRLTDQPHN NRDIELNRAK RATTNPSPPA QAPSENLTLR DVQPLKDSAL HYQYVLIDLQ SARLPVYTRK TFERELALEW IIPDAEEA.

In some embodiments, the viruses, viral polypeptides, and/or analogs thereof are derived from a protein sequence of the Pecluvirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to a capsid protein of a virus in the Pecluvirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to SEQ ID NO:4: MANISEVRRG GGHYSIASWR NHVIKQNLNH DWWIRSDRWA QLLADLRAVN YEVNSSRSEV ANIINRVPKD LPADPSARFP GVVGTPGETN YSLVYYVRVE APVREKFLGI IAAADQGKSR DVEVGRPSAP SVASGAGNQA IVPVRGVNAI RDQQPLRDGS LSFRYKLVDV ELASVTQFDQ VLFEETFQIT WIEKDASTKT TPTASTNTSP TGVAPGDPSN.

In some embodiments, the viruses, viral polypeptides, and/or analogs thereof are derived from a protein sequence of the Pomovirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to a capsid protein of a virus in the Pomovirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to SEQ ID NO:5: MTAAMEPHYA VFSNKMAKYA AAHPFIKYNE LSETVKSWMQ TRTSVMEHVN FVLGSAANLG TRGFFSRNVR FGMTNVNGDN LYADLGYLPF QNLLNALTIV LGAVGGRGKL RRNPKGESSK AAATEQINGG SDGQLNIAHC IMDINQVMSD PTILQNAVYS QSTFEEAHGL AWVYKPTA.

In some embodiments, the viruses, viral polypeptides, and/or analogs thereof are derived from a protein sequence of the Tobravirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to a capsid protein of a virus in the Tobravirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to SEQ ID NO: 6: MAMYDDEFDT KASDLTFSPW VEVENWKDVT TRLRAIKFAL QADRDKIPGV LSDLKTNCPY SAFKRFPDKS LYSVLSKEAV IAVAQIQSAS GFKRRADEKN AVSGLVSVTP TQISQSASSS AATPVGLATV KPPRESDSAF QEDTFSYAKF DDASTAFHKA LAYLEGLSLR PTYRRKFEKD MNVKWGGSGS APSGAPAGGS SGSAPPTSGS SGSGAAPTPP PNP.

In some embodiments, the viruses, viral polypeptides, and/or analogs thereof are derived from a protein sequence of the Inoviridae family. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to a capsid protein of a virus in the Inoviridae family. In some embodiments, the viruses, viral polypeptides, and/or analogs thereof are derived from a protein sequence of the Inovirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to a capsid protein of a virus in the Inovirus genus. In some embodiments, the viruses, viral polypeptides, and/or analogs thereof comprise at least 70%, 75%, 80%, 85%, 90%, 91%, 92%, 93%, 94%, 95%, 96%, 97%, 98%, or 99% sequence identity to SEQ ID NO:7: MKKSLVLKAS VAVATLVPML SFAAEGDDPA KAAFNSLQAS ATEYIGYAWA MVVVIVGATI GIKLFKKFTS KAS.

In some embodiments, an array or system disclosed herein comprises no less than 1.0 cm squared of sensing surface area on the solid support. In some embodiments, an array or system disclosed herein comprises no less than 1.2 cm squared of sensing surface area on the solid support. In some embodiments, an array or system disclosed herein comprises no less than 1.4 cm squared of sensing surface area on the solid support. In some embodiments, an array or system disclosed herein comprises no less than 1.6 cm squared of sensing surface area on the solid support. In some embodiments, an array or system disclosed herein comprises no less than 1.8 cm squared of sensing surface area on the solid support. In some embodiments, an array or system disclosed herein comprises no less than 2.0 cm squared of sensing surface area on the solid support. In some embodiments, an array or system disclosed herein comprises no less than 2.4 cm squared of sensing surface area on the solid support. In some embodiments, an array or system disclosed herein comprises no less than 2.8 cm squared of sensing surface area on the solid support. In some embodiments, an array or system disclosed herein comprises no less than 3.0 cm squared of sensing surface area on the solid support. In some embodiments, an array or system disclosed herein comprises no less than 3.3 cm squared of sensing surface area on the solid support. In some embodiments the total surface area of the sensor or device is no less than 3.0 cm squared on a solid support with width of 7 nm and a length of 7 nm.

The invention further relates to an array of polypeptides, the array comprising: a solid support and a plurality of viruses and/or viral proteins and/or analogs thereof, wherein the viruses and/or viral proteins and/or analogs thereof are immobilized to the solid support at least one addressable location of the array, and wherein the density of a plurality of viruses and/or viral proteins and/or analogs thereof is at least about 1, 2, 3, 3.1, 3.2, 3.3, 3.4, 3.5, 3.6, 3.7, 3.8 3.9, 3.91, 3.92, 3.93, 3.94, 3.95, 3.96, 3.97, 3.98 and 4*10⁹ virus/mm².

The invention further relates to an array of polypeptides, the array comprising: a solid support and a plurality of viruses and/or viral proteins and/or analogs thereof, wherein the viruses and/or viral proteins and/or analogs thereof are immobilized to the solid support at least one addressable location of the array, and wherein the biodetection efficiency is from about 2 to about 8 times the biodetection efficiency of an array that doe not comprise one or a combination of any of the disclosed: (i) plurality of viruses; (ii) plurality of viral proteins; and/or (iii) plurality of analogs thereof. In some embodiments, the biodetection efficiency is from about 2 to about 9 times the biodetection efficiency of an array that doe not comprise one or a combination of any of the disclosed: (i) plurality of viruses; (ii) plurality of viral proteins; and/or (iii) plurality of analogs thereof the biodetection efficiency is from about 3 to about 9 times the biodetection efficiency of an array that doe not comprise one or a combination of any of the disclosed: (i) plurality of viruses; (ii) plurality of viral proteins; and/or (iii) plurality of analogs thereof the biodetection efficiency is from about 4 to about 9 times the biodetection efficiency of an array that doe not comprise one or a combination of any of the disclosed: (i) plurality of viruses; (ii) plurality of viral proteins; and/or (iii) plurality of analogs thereof the biodetection efficiency is from about 5 to about 9 times the biodetection efficiency of an array that doe not comprise one or a combination of any of the disclosed: (i) plurality of viruses; (ii) plurality of viral proteins; and/or (iii) plurality of analogs thereof the biodetection efficiency is from about 6 to about 9 times the biodetection efficiency of an array that doe not comprise one or a combination of any of the disclosed: (i) plurality of viruses; (ii) plurality of viral proteins; and/or (iii) plurality of analogs thereof. the biodetection efficiency is from about 7 to about 9 times the biodetection efficiency of an array that doe not comprise one or a combination of any of the disclosed: (i) plurality of viruses; (ii) plurality of viral proteins; and/or (iii) plurality of analogs thereof the biodetection efficiency is from about 8 to about 9 times the biodetection efficiency of an array that doe not comprise one or a combination of any of the disclosed: (i) plurality of viruses; (ii) plurality of viral proteins; and/or (iii) plurality of analogs thereof.

The invention further relates to an array of polypeptides, the array comprising: a solid support and a plurality of viruses and/or viral proteins and/or analogs thereof, wherein the viruses and/or viral proteins and/or analogs thereof are immobilized to the solid support at least one addressable location of the array, and wherein the plurality of viruses and/or viral proteins and/or analogs thereof, wherein the viruses and/or viral proteins and/or analogs thereof are immobilized to the solid support at least one addressable location of the array, and wherein the signal to noise ratio is from about 1 to about 2, calculated by the signal obtained from a probe to a target molecule or substance as compared to the signal obtained by detection of affinity using a non-specific (or control) probe. In some embodiments, the signal to noise ratio is from about 1.1 to about 1.5 In some embodiments, the signal to noise ratio is from about 1.1 to about 1.3. In some embodiments, the signal to noise ratio is from about 1.11 to about 1.20. In some embodiments, the signal to noise ratio is about 1.20.

The invention relates to an array of polypeptides, the array comprising: a solid support and a plurality of association sets, wherein each association set comprises two or more different polypeptides comprising a polypeptide sequence associated with the extracellular matrix or a functional fragment thereof, and wherein the array further comprises one or a plurality of mammalian cells. In some embodiments, the one or a plurality of mammalian cells contains at least one lung cell.

The invention further provides an array or kit comprising at least on array or device disclosed herein. In some embodiments, the device or array comprises any of the disclosed combinations of viruses, viral proteins, analogs thereof and at least a first container that comprises a preservation solution, an aqueous buffer created to preserve a test sample or target molecule. In some embodiments, the kit comprises an element or swab for contacting a test solution or surface and collecting a test sample comprising or suspected of comprising a target molecule. In some embodiments, the kit comprises at least one container comprising any of the probes described herein.

The invention relates to an array of polypeptides, the array comprising: a solid support and a plurality of vertically oriented viruses, viral proteins, and or analogs thereof immobilized on the solid support, wherein, if the array comprises viral proteins and/or analogs thereof, the viral protein is TMV capsid or an analog thereof. In some embodiments, the viruses, viral proteins and or analogs thereof are immobilized to the solid support via passive electrostatic non-covalent binding. In some embodiments, at least a portion of the surface area of the a plurality of viruses and/or viral proteins and/or analogs thereof are coated with nickel at a depth of from about 40 nm to about 60 nm. In some embodiments, at least a portion of the surface area of the a plurality of viruses and/or viral proteins and/or analogs thereof are coated with gold at a depth of from about 40 nm to about 60 nm.

The invention provides a system comprising: an array of polypeptides, the array comprising: a solid support and a plurality of vertically oriented viruses, viral proteins, and/or analogs thereof immobilized on the solid support, wherein a portion of the viruses, viral proteins, and or analogs thereof immobilized on the solid support are coated with at least one or two layers metal; and at least one voltmeter and/or ammeter. In some embodiments, the system further comprises at least one or a plurality of circuits in operable connection to the solid support and the at least one voltmeter and/or ammeter. In some embodiments, the system further comprises at least one or a plurality of wires operably connecting the solid support and the at least one voltmeter and/or ammeter. In some embodiments, the system further comprises at least one test sample.

The invention also provides a kit comprising: an array of polypeptides, the array comprising: a solid support and a plurality of one or a combination of: a plurality of vertically oriented viruses; and/or a plurality of viral proteins and/or analogs thereof, wherein at least a portion of the plurality of viruses, viral proteins, or analogs thereof are derived from Virgaviridae family of viruses or a genus thereof. In some embodiments, the at least a portion of the plurality of viruses, viral proteins, or analogs thereof are derived from TMV. In some embodiments, the kit further comprises at least one of the following: a swab or element, a test sample, an antibody, a volume of fluorescent stain or dye, a probe, and a set of instructions, optionally accessible remotely through an electronic medium.

The invention further provides a method of identifying an association signature of a cell sample comprising: contacting a cell sample to an array or system disclosed herein; and determining a quantity of cells bound to one or a plurality of association sets. In some embodiments, the cell sample contains at least one cell from a biopsy.

### BRIEF DESCRIPTION OF THE DRAWINGS

FIG. 1 depicts a self-assembled TMV-1 cys molecules coated with nickel and gold layers on the surface of planar electrode. The top section of FIG. 1 depicts a cross section schematic of the sensor. The bottom section of FIG. 1 depicts a scanning electron micrograph of the sensor surface.

FIG. 2 depicts a schematic of the TMV coatings on the left-hand side (identified as section A). FIG. 2 also depicts a transmission electron microscopy analysis of the TMV-1 cys coated with nickel and gold layers in which EDS profiles of nickel (bottom) and gold (top) are overlayed on the image of the coated virus on the right-hand side (identified as section B).

FIG. 3 depicts cyclic voltammograms of the (A) planar electrode (without virus), (B) TMV modified electrode, (C) Ni/Au modified electrode, and (D) TMV/Ni/Au modified electrode following introduction of ssDNA NRGJ probe (black solid), non-complementary ssDNA (grey dashed line), and complementary NRGJ target ssDNA on Ni/Au modified electrode (grey dashed-dot). Arrow indicates current reduction upon DNA hybridization.

FIG. 4 depicts cyclic voltammograms of the planar electrode modified with either the TMV or the Ni/Au electroless deposition.

### DETAILED DESCRIPTION OF THE INVENTION

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “addressable location” as used herein means a discrete surface area or position on a solid support onto which one or a plurality of viruses or viral proteins are immobilized or absorbed such that exposure of the one or plurality of viruses or viral proteins optionally coated with one or more metals to a sample comprising a substance and a for a sufficient time period results in association between the virus or viral protein or metto the. In some embodiments, the one or plurality of addressable locations of the array is spotted manually by a pipet or automatically by a robotic device.

As used herein, the terms “attach,” “attachment,” “adhere,” “adhered,” “adherent,” or like terms generally refer to immobilizing or fixing, for example, a group, a compound or virus, to a surface, such as by physical absorption, chemical bonding, and like processes, or combinations thereof.

The terms “association value” as used herein means a single quantitative value that can be used as a criterion for whether a particular sample comprises or does not comprise a particular quantity of substance such that, when normalized against a quantitative value calculated for a control sample, the association value can be used in a predictive fashion for the diagnosis, prognosis, clinical treatment plan of a subject or the relative degree of contamination in a fluid or on a surface. In some embodiments, the association value means a single quantitative value that can be used as a criterion for how tightly or how loosely a particular substance does or does not associate (or bind) to a particular quantity of material or probe or surface on any of the disclosed device, sensors, or compositions disclosed herein such that, when normalized against a comparably calculated quantitative value for the control, the association value can be used in a predictive model for the diagnosis, prognosis, or clinical treatment plan of a subject or in the contamination assessment of a fluid sample or surface. In some embodiments, the quantitative value is calculated by combining quantitative data regarding the association of a substance to one or a plurality of viruses or viral proteins through an interpretation function or algorithm described herein. In some embodiments, the subject is suspected of having, is at risk of developing, or has been diagnosed with a disease or disorder. In some embodiments, the subject is suspected of having, is at risk of developing, or has been diagnosed with a psychotic disorder or a pathogen infection.

As used herein, the terms “biopsy” means a cell sample, collection of cells, or tissue removed from a subject or patient for analysis. In some embodiments, the biopsy is a bone marrow biopsy, punch biopsy, endoscopic biopsy, needle biopsy, shave biopsy, incisional biopsy, excisional biopsy, or surgical resection.

As used herein, “conservative” amino acid substitutions may be defined as set out in Tables A, B, or C below. Viral proteins and analogs thereof include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table A.

TABLE A
Conservative Substitutions I

| Side Chain Characteristics | | Amino Acid |
|---|---|---|
| Aliphatic | Non-polar | GAPILVF |
| | Polar—uncharged | CSTMNQ |
| | Polar—charged | DEKR |
| Aromatic | | HFWY |
| Other | | NQDE |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| Non-polar (hydrophobic) | |
| Aliphatic: | ALIVP. |
| Aromatic: | FWY |
| Sulfur-containing: | M |
| Borderline: | GY |
| Uncharged-polar | |
| Hydroxyl: | STY |
| Amides: | NQ |
| Sulfhydryl: | C |
| Borderline: | GY |
| Positively Charged (Basic): | KRH |
| Negatively Charged (Acidic): | DE |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides comprising polypeptide sequences a described herein are intended to include polypeptides bearing one or more insertions, deletions, or substitutions, or any combination thereof, of amino acid residues as well as modifications other than insertions, deletions, or substitutions of amino acid residues.

As used herein the terms “electronic medium” mean any physical storage employing electronic technology for access, including a hard disk, ROM, EEPROM, RAM, flash memory, nonvolatile memory, or any substantially and functionally equivalent medium. In some embodiments, the software storage may be co-located with the processor implementing an embodiment of the invention, or at least a portion of the software storage may be remotely located but accessible when needed via the internet or portable storage memory device.

As used herein, the term “prognosing” means determining the probable course and outcome of a disease.

As used herein, the term “functional fragment” means any portion of a polypeptide to which the polypeptide relates that is of a sufficient length and has a sufficient structure to confer a biological affect that is similar or substantially similar to the full-length polypeptide upon which the fragment is based. In some embodiments, the fragment is a fragment of any of the amino acid sequences disclosed herein either individually or, when concatamerized or self-assembling, as a subunit as part of a larger filamentous particle. In some embodiments, the functional fragment is a fragment of TMV capsid protein and has a length of at least about 10 amino acids. In some embodiments, the fragment is a fragment of TMV capsid protein and has a length of at least about 50 amino acids. In some embodiments, the fragment is a fragment of TMV capsid protein and has a length of at least about 100 amino acids. In some embodiments, the fragment is a fragment of TMV capsid protein and has a length of at least about 150 amino acids. In some embodiments, the fragment is a fragment of TMV capsid protein and has a length of at least about 200 amino acids. In some embodiments, the fragment is a fragment of TMV capsid protein and has a length of at least about 250 amino acids. In some embodiments, the fragment is a fragment of TMV capsid protein and has a length of at least about 300 amino acids. In some embodiments, the fragment is a fragment of TMV capsid protein and has a length of at least about 350 amino acids. In some embodiments, the fragment is a fragment of TMV capsid protein and has a length of at least about 400 amino acids. In some embodiments, the fragment is a fragment of TMV capsid protein and has a length of at least about 450 amino acids. In some embodiments, the fragment is a fragment of TMV capsid protein and has a length of at least about 500 amino acids. In some embodiments, the fragment is a fragment of TMV capsid protein and has a length of at least about 550 amino acids. In some embodiments, the fragment is a fragment of TMV capsid protein and has a length of at least about 600 amino acids. In some embodiments, the fragment is a fragment of TMV capsid protein and has a length of at least about 650 amino acids. In some embodiments, the fragment is a fragment of TMV capsid protein and has a length of at least about 700 amino acids. In some embodiments, the fragment is a fragment of TMV capsid protein and has a length of at least about 750 amino acids. In some embodiments, the fragment is a fragment of TMV capsid protein and has a length of at least about 800 amino acids. In some embodiments, the fragment is a fragment of TMV capsid protein and has a length of at least about 850 amino acids. In some embodiments, the fragment is a fragment of TMV capsid protein and has a length of at least about 900 amino acids. In some embodiments, the fragment is a fragment of TMV capsid protein and has a length of at least about 950 amino acids. In some embodiments, the fragment is a fragment of TMV capsid protein and has a length of at least about 1000 amino acids. In some embodiments, the fragment is a fragment of TMV capsid protein and has a length of at least about 1050 amino acids.

The term “analog” refers to any polypeptide that is structurally similar to a naturally occurring full-length protein and that shares the biochemical or biological activity of the naturally occurring full-length protein upon which the analog is based. Various references disclose modification of polypeptides by polymer conjugation or glycosylation. The term analog includes polypeptides conjugated to a polymer such as PEG and may be comprised of one or more additional derivitizations of cysteine, lysine, or other residues. In addition, analogs of the instant invention may comprise a linker or polymer, wherein the amino acid to which the linker or polymer is conjugated may be a non-natural amino acid, or may be conjugated to a naturally encoded amino acid utilizing techniques known in the art such as coupling to lysine or cysteine. encompasses the conventional and well-known naturally occurring amino acids, as well as all synthetic variations and derivatives thereof. In some embodiments, the analogs comprise alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine substitutions. In some embodiments, analogs comprise N-methylated α-amino acids, hydroxylated amino acids, and aminoxy acids. In some embodiments, analogs comprise N-alkyl amino acids (such as N-methyl glycine), hydroxylysine, 3-hydroxyproline, 4-hydroxyproline, nor-valine, nor-leucine, and ornithine. The terms “amino acid” refer to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs including non-natural analogs.

The terms “longitudinal axis” refers to the an axis of the filamentous protein, capsid protein or collection running lengthwise of the virus or viral protein or analog thereof. In the case of TMV, for instance, if the TMV lengthwise on a z axis (with its side of about 300 nm running up the z axis) the longitudinal axis is an axis that runs on through the longest length of the virus or filamentous protein comprising subunits or an axis substantially aligned thereto.

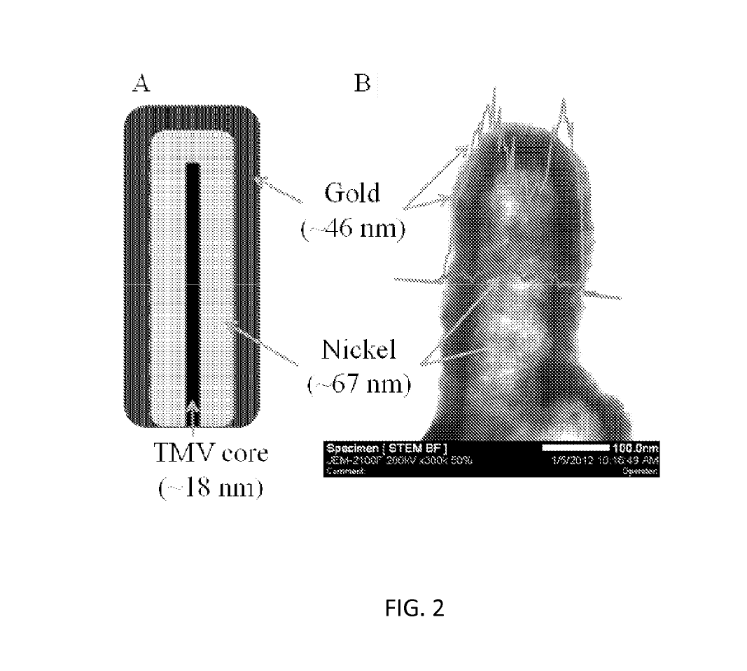

The terms “successive layers” as defined here means a layer of material that is immediately adjacent to another layer of material. For example, in relation to a sensor, the sensor, device, or array disclosed herein may contain at least 3 successive layers of material deposited on a solid support or electrode. The layers as depicted in FIG. 2 are a virus or viral core protein, a layer of nickel, and another layer of gold. Each of the layers are immediately adjacent to the layer of material they are preceded by in a list.

The terms “functional fragment” means any portion of a polypeptide or nucleic acid sequence from which the respective full-length polypeptide or nucleic acid relates that is of a sufficient length and has a sufficient structure to confer a biological affect that is similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence homology to the wild-type sequence upon which the sequence is derived. The present invention also comprises functional fragments of amino acid nucleotide sequences that encode a polypeptide capable of. In some embodiments, the functional fragment are amino acid fragments selected from at least one of the various encoding amino acid sequences of the present invention, including SEQ ID NO: 1 and can be any of the following described amino acid fragments, as it applies to the specific amino acid sequences provided herein. In some embodiments, amino acid fragments can comprise 30 or more, 45 or more, 60 or more, 75 or more, 90 or more, 120 or more, 150 or more, 180 or more, 210 or more, 240 or more, 270 or more, 300 or more, 360 or more, 420 or more, 480 or more, 540 or more, 600 or more, 660 or more, 720 or more, 780 or more, 840 or more, 900 or more, 960 or more, 1020 or more, 1080 or more, 1140 or more, 1200 or more, 1260 or more, 1320 or more, 1380 or more, 1440 or more, 1500 or more, 1560 or more, 1620 or more, 1680 or more, or 1740 or more, 1800 or more, 2000 or more, 2100 or more, 2200 or more, 2300 or more, 2400 or more, 2500 or more, 2600 or more, 2700 or more, 2800 or more, 2900 or more, 3000 or more, 4000 or more, 4500 or more, 5000 or more, 5500 or more, 6000 or more, 6500 or more, 7000 or more, 7500 or more, 8000 or more, 8500 or more, 9000 or more, 9500 or more, 10000 or more, 10100 or more amino acids. In some embodiments, amino acid fragments can comprise coding sequences for accessory proteins such as known ligands to tumor associated antigens expressed on the surface of tumor cells. In some embodiments, amino acid fragments can comprise fewer than 60, fewer than 75, fewer than 90, fewer than 120, fewer than 150, fewer than 180, fewer than 210, fewer than 240, fewer than 270, fewer than 300, fewer than 360, fewer than 420, fewer than 480, fewer than 540, fewer than 600, fewer than 660, fewer than 720, fewer than 780, fewer than 840, fewer than 900, fewer than 960, fewer than 1020, fewer than 1080, fewer than 1140, fewer than 1200, fewer than 1260, fewer than 1320, fewer than 1380, fewer than 1440, fewer than 1500, fewer than 1560, fewer than 1620, fewer than 1680, or fewer than 1740, fewer than 1800, fewer than 1900, fewer than 2000, fewer than 2100, fewer than 2200, fewer than 2300, fewer than 2400, fewer than 2500, fewer than 2600, fewer than 2700, fewer than 2800, fewer than 2900, fewer than 3000, fewer than 4000, fewer than 5000, fewer than 6000, fewer than 7000, fewer than 8000, fewer than 9000, or fewer than 10000 amino acids. In some embodiments, the analogs are functional fragments of SEQ ID NO:1-7 and include one or more amino acid derivative, either naturally occurring or non-naturally occurring. In some embodiments, the functional fragments are amino acid fragments of SEQ ID NO:1-7 and include more than 5, 10, 15, 20, 25, or 30 nucleic acid or amino acid derivatives.

As used herein, the term “psychotic disorder” refers to an abnormal condition in which the subject's mental state becomes impaired. Examples of psychotic disorders include: affective mood disorders, bipolar disorder, borderline personality disorder, brief psychotic disorder, brief reactive psychosis, chronic hallucinatory psychosis, induced delusional disorder, paranoid personality disorder, persistent delusional disorder, post-traumatic stress disorder, schizoaffective disorder, schizoid personality disorder, schizophrenia, schizophreniform disorder, schizotypal disorder, and types of severe depression.

As used herein, the term “antibody” refers to any immunoglobulin, whether natural or wholly or partially synthetically produced. In some embodiments, an antibody is a complex comprised of 4 full-length polypeptide chains, each of which includes a variable region and a constant region, e.g., substantially of the structure of an antibody produced in nature by a B cell. In some embodiments, an antibody is a single chain. In some embodiments, an antibody is cameloid. In some embodiments, an antibody is an antibody fragment. In some embodiments, an antibody is chimeric. In some embodiments, an antibody is bi-specific. In some embodiments, an antibody is multi-specific. In some embodiments, an antibody is monoclonal. In some embodiments, an antibody is polyclonal. In some embodiments, an antibody is conjugated (i.e., antibodies conjugated or fused to other proteins, radiolabels, cytotoxins). In some embodiments, an antibody is a human antibody. In some embodiments, an antibody is a mouse antibody. In some embodiments, an antibody is a rabbit antibody. In some embodiments, an antibody is a rat antibody. In some embodiments, an antibody is a donkey antibody.

An “array”, as that term is used herein, typically refers to an arrangement of entities (in some embodiments, a plurality of viruses) in spatially discrete locations of a solid support with respect to one another, and usually in a format that permits simultaneous exposure of the arranged entities to potential target samples or substances or other reagents, substrates, etc. In some embodiments, an array comprises entities arranged in spatially discrete locations on a solid support. In some embodiments, spatially discrete locations on an array are arranged in a regular pattern with respect to one another (e.g., in a grid). In some embodiments, the array comprises a confluent layer of a plurality of viruses or viral proteins that are directionally immobilized to the spatially discrete locations on the solid support.

The term “binding partner” is used herein to refer to any two entities that specifically bind with each other in a given context. In some embodiments, binding is specific in that a binding agent has a greater affinity for its target binding partner than for other potential binding partners in its environment. Binding partners may be of any chemical type. In some embodiments, binding partners are polypeptides or nucleic acid molecules In some embodiments, binding partners are antibodies with a specific affinity for a target molecule or substance. In some embodiments, binding partners are carbohydrates.

As is used herein, the term “comparable” is used to refer to two entities that are sufficiently similar to permit comparison, but differing in at least one feature.

As used herein, the term “kit” refers to a set of components provided in the context of a delivery system for delivering materials. Such delivery systems may include, for example, systems that allow for storage, transport, or delivery of various diagnostic or therapeutic reagents (e.g., oligonucleotides, enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to delivery systems comprising two or more separate containers that each contain a subportion of total kit components. Containers may be delivered to an intended recipient together or separately. For example, a first container may contain a petri dish for use in a cell culture assay, while a second container may contain cells. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a subportion of total kit components are included in the term “fragmented kit.” In contrast, a “combined kit” refers to a delivery system containing all components in a single container (e.g., in a single box housing each of the desired components). The term “kit” includes both fragmented and combined kits.

The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having the complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein.

As used herein, the term “sample” refers to a biological sample obtained or derived from a source of interest, as described herein. In some embodiments, a source of interest comprises an organism, such as an animal or human. In some embodiments, a biological sample comprises biological tissue or bodily fluid. In some embodiments, a biological sample may be or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, the sample may be a water sample or food sample. In some embodiments, the sample or test sample may be reconstituted in solution after swabbing a surface or food or collecting water or other liquid for detection of contaminants. In some embodiments, the cell sample is isolated from a subject suspected of having, being at risk for developing, or diagnosed with a psychotic disorder. In some embodiments, the cell sample is isolated from a subject suspected of having, being at risk for developing, or diagnosed with schizophrenia.

The term “nucleic acid” refers to a molecule comprising two or more linked nucleotides. “Nucleic acid” and “nucleic acid molecule” are used interchangeably and refer to oligoribonucleotides as well as oligodeoxyribonucleotides. The terms also include polynucleosides (i.e., a polynucleotide minus a phosphate) and any other organic base containing nucleic acid. The organic bases include adenine, uracil, guanine, thymine, cytosine and inosine. The nucleic acids may be single or double stranded. The nucleic acid may be naturally or non-naturally occurring. Nucleic acids can be obtained from natural sources, or can be synthesized using a nucleic acid synthesizer (i.e., synthetic). Isolation of nucleic acids are routinely performed in the art and suitable methods can be found in standard molecular biology textbooks. (See, for example, Maniatis' Handbook of Molecular Biology.) The nucleic acid may be DNA or RNA, such as genomic DNA, mitochondrial DNA, mRNA, cDNA, rRNA, miRNA, PNA or LNA, or a combination thereof, as described herein. Non-naturally occurring nucleic acids such as bacterial artificial chromosomes (BACs) can also be used in accordance with some aspects of this invention.

As used herein, the term “probe” refers to any molecule that binds to or intercalates with a target molecule or substance, either covalently or non-covalently. In some embodiments, the probes include probe sets which include one or more probes that bind a target molecule or substance. The term “probe set” is sometime interchangeable for a panel of two or more probes that allow the detection of one or more target molecules and/or substances. In some embodiments the probe or probes are fluorescently labeled. In some embodiments, each fluorescently labeled probe is specific for at least one target molecule and/or substance. In one embodiment of the invention, the panel of probes detects relies upon the presence or absence of a product of a reduction/oxidation reaction so that upon a binding event positive or negative charge is contacted to the electrode. The increase in charge can be detected by the electrode, in the case of the sensor or device disclosed herein, to which the viruses, viral protein or analogs thereof are immobilized.

In some embodiments, the sensor, device, system, or composition disclosed herein comprises one or more electrode, or substance with electroconductive properties. In some embodiments, the one or more electrodes transmit current variation generated by the reaction between the probe and its binding partner or the presence of electrochemically active analytes or target molecules (such as clozapine). In some embodiments, the electrodes comprise metal. In some embodiments, the electrodes comprise a carbon scaffold upon which a metal is deposited. In some embodiments, the electrodes comprise a carbon scaffold of carbon nanotubes upon which viruses or viral proteins and/or analogs thereof are deposited. Electrode structures which are suitable for the present invention and methods for the production of such structures have already been suggested in for other purposes. In this regard, reference is made to U.S. Pat. No. 6,645,359 and its content is incorporated herein by reference in its entirety. Electrodes or Electrically conductive tracks are created or isolated on first surface. Tracks represent the electrodes of a sensor. As used herein, the phrase “electrode set” is a set of at least two electrodes, for example 2 to 200, or 3 to 20, electrodes. These electrodes may, for example, be a working (or measuring) electrode and an auxiliary electrode. In some embodiments, tracks cooperate to form an interdigitated electrode array positioned within the periphery of recesses and leads that extend from array and between recesses toward end. Tracks are constructed from electrically conductive materials. Non-limiting examples of electrically-conductive materials include aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (such as highly doped polycrystalline silicon), silver, tantalum, tin, titanium, tungsten, uranium, vanadium, zinc, zirconium, mixtures thereof, and alloys, oxides, or metallic compounds of these elements. Track can be made of include gold, platinum, palladium, iridium, or alloys of these metals, since such noble metals and their alloys are unreactive in biological systems. in some embodiments the solid support is an electrode or group of electrodes or tracks. In some embodiments, the track is a working electrode made of silver and/or silver chloride, and track is an auxiliary electrode that is also made of silver and/or silver chloride and is substantially the same size as the working electrode.

Tracks are isolated from the rest of the electrically conductive surface by laser ablation. Techniques for forming electrodes on a surface using laser ablation are known. Techniques for forming electrodes on a surface using laser ablation are known. See, for example, U.S. patent application Ser. No. 09/411,940, filed Oct. 4, 1999, and entitled “LASER DEFINED FEATURES FOR PATTERNED LAMINATES AND ELECTRODE”, the disclosure of which is expressly incorporated herein by reference. Tracks are preferably created by removing the electrically conductive material from an area extending around the electrodes. Therefore, tracks are isolated from the rest of the electrically-conductive material on a surface by a gap having a width of about 5 μm to about 500 μm, preferably the gap has a width of about 100 μm to about 200 μm. Alternatively, it is appreciated that tracks may be created by laser ablation alone on bottom substrate. Further, tracks may be laminated, screen-printed, or formed by photolithography. Multi-electrode arrangements are also possible in accordance with this disclosure.

Reference electrode: As will be understood from context, a “reference electrode’ or control electrode is an electrically conductive support such as an electrode placed in a circuit with an at least one electrically conductive support comprising hydrogel and/or immobilized enzymes disclosed herein, to permit a relevant comparison of voltage difference between the reference or control electrode and the at least one electrically conductive support comprising hydrogel and/or immobilized enzymes disclosed herein. Any device, sensor or array disclosed herein may comprise at least two electrodes, one electrode for testing the affinity of a target molecule and one reference electrode.

In some embodiments, any array, device, sensor, or composition disclosed herein comprise at least one or a combination of probes the detect one or a plurality of heavy metals, radioisotopes, and DNA from a pathogenic microorganism. In some embodiments, any array, device, sensor, or composition disclosed herein comprise at least one or a combination of the following probes, optionally immobilized on a discrete portion of the gold layer of the electrode:

For heavy metal, any antibody or probe disclosed in U.S. Pat. No. 5,908,790 or EP 0173629B1, which are incorporated by reference in their entireties; wherein if the disclosed methods include a step of providing a salt or buffer in conjunction with its use, any of the methods disclosed herein may also comprise the step of providing or exposing the device, sensor, or array with the requisite salt or buffer.

For radioisotope: any antibody or probe used to identify a radioisotope may be immobilized to the surface of the at least one metal layer (in some embodiments, the gold layer). U.S. Pat. No. 7,723,114 discloses diesterified methanediphosphonic acid as a way to capture certain types of radioisotopes. U.S. Pat. No. 7,923,692B2 discloses ALIQUAT® 336 as a ligand to separate 99Tc. These patents and patent applications are incorporated by reference in their entireties.

For detection of pathogenic bacteria, any antibody may be used to detect the presence of the E. coli pathogen. in some embodiments, antibodies disclosed in US 20130130364 may be immobilized to a gold layer on the device, sensor, or array disclosed herein for capture of antigens associated with the pathogenic bacteria.

Some aspects of this invention relate to the use of nucleic acid derivatives as a full or partial component of a probe. The use of certain nucleic acid derivatives may increase the stability of the nucleic acids of the invention by preventing their digestion, particularly when they are exposed to biological samples that may contain nucleases. As used herein, a nucleic acid derivative is a non-naturally occurring nucleic acid or a unit thereof. Nucleic acid derivatives may contain non-naturally occurring elements such as non-naturally occurring nucleotides and non-naturally occurring backbone linkages. Nucleic acid derivatives according to some aspects of this invention may contain backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof. The backbone composition of the nucleic acids may be homogeneous or heterogeneous. Nucleic acid derivatives according to some aspects of this invention may contain substitutions or modifications in the sugars and/or bases. For example, some nucleic acid derivatives may include nucleic acids having backbone sugars which are covalently attached to low molecular weight organic groups other than a hydroxyl group at the 3′ position and other than a phosphate group at the 5′ position (e.g., an 2′-O-alkylated ribose group). Nucleic acid derivatives may include non-ribose sugars such as arabinose. Nucleic acid derivatives may contain substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. In some embodiments, a nucleic acid may comprise a peptide nucleic acid (PNA), a locked nucleic acid (LNA), DNA, RNA, or a co-nucleic acids of the above such as DNA-LNA co-nucleic acid.

“Sequence homology” or “sequence identity” are used herein interchangeably for nucleotides and amino acids sequences determined using FASTA, BLAST and Gapped BLAST (Altschul et al., Nuc. Acids Res., 1997, 25, 3389, which is incorporated herein by reference in its entirety) and PAUP* 4.0bIO software (D. L. Swofford, Sinauer Associates, Massachusetts). Briefly, the BLAST algorithm, which stands for Basic Local Alignment Search Tool is suitable for determining sequence similarity (Altschul et al., J. MoI. Biol, 1990, 215, 403-410, which is incorporated herein by reference in its entirety). Software for performing BLAST analyses is publicly available through the National Center for Biotechnology Information (http://www.ncbi.nlm.nih.gov). One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, preferably less than about 0.1, more preferably less than about 0.01, and most preferably less than about 0.001. “Percentage of similarity” or percentage of sequence identity” can be calculated using PAUP* 4.0bIO software (D. L. Swofford, Sinauer Associates, Massachusetts). The average similarity of the consensus sequence is calculated compared to all sequences in the phylogenic tree.

The term “subject” is used throughout the specification to describe an animal to whom treatment with the compositions according to the present invention is provided or administered. For treatment of those conditions which are specific for a specific subject, such as a human being or such as a mammal, the term “patient” may be interchangeably used. In some instances in the description of the present invention, the term “patient” will refer to human patients. In some embodiments, the subject may be a mammal to whom the present invention is provided or administered. In some embodiments, the subject may be a domesticated mammal to whom the present invention is provided or administered such as a horse, dog, cat, pig, cow, goat, sheep, llama, or other non-human animal. In some embodiments, the subject is non-human. In some embodiments, the subject is a mammal suspected of having an infection of a pathogenic microorganism. In some embodiments, the subject is an animal diagnosed with and/or suspected of having been infected by a pathogenic microorganism.

The term “substance” as used herein is defined as any form of matter, including any element or groups of chemical elements that have physical mass. In some embodiments, the substance is an atom or combination of atoms. In some embodiments, the substance is an ion or combination of ions. In some embodiments, the substance is a heavy metal or combination of heavy metals. In some embodiments, the substance is a radioisotope or combination of radioisotopes. In some embodiments, the substance is a target molecule or combination of target molecules. In some embodiments, the substance is a as any form of matter that is detectable by any array, device, or composition disclosed herein.

The term “target molecule” as used herein is defined as a molecular compound with at least one chemical bond between at least two elements. In some embodiments, the target molecule is an inorganic compound. In some embodiments, the target molecule is an organic compound. In some embodiments, the target molecule is or comprises a nucleotide. In some embodiments, the target molecule is a complementary strand of a nucleic acid sequence or nucleic acid molecule. In some embodiments, the target molecule is a protein or a hybriod molecule comprising at least one peptide and at least one nucleotide. In some embodiments, the target molecule is a molecular compound with at least one chemical bond between at least two elements and is the target to which a probe or probes on the device, sensor, array, or composition bind. In some embodiments, the target molecule is a ssDNA complementary to the ssDNA SNP8NRG243177. In some embodiments, the target molecule is a polypeptide that has an binds to any antibody or probe on the device, sensor, array, or composition disclosed herein.

The term “testing surface” as used herein is defined as a surface of any substrate that comprises or is thought to comprise a target molecule or substance. In some embodiment, the testing surface is a laboratory bench, surface of a piece of food, or surface area upon which hazardous materials are transported or used (including water), In some embodiments, the testing surface is the surface from which a test sample, substance or target molecule is taken for use or characterization in any of the devices, array, sensors, or compositions described herein. In some embodiments, the surface comprises or is suspected of comprising one or more target molecule or target substance. In some embodiments, the testing surface comprises metal ions or one or more layers of metal ions. In some embodiments, the testing surface comprises a contaminant, pathogen, or substance hazardous to the health of a subject. In some embodiments, the testing surface is water, a food source, or a testing solution that comprises a swab or element comprising a contaminant, pathogen, or substance hazardous to the health of a subject.

The term “inert surface” as used herein is defined as a surface, coating, or film on any array, device, or composition disclosed herein that is not capable of binding to and/or interacting with a target molecule.

The present invention relates to compositions comprising any one or combination of viruses, viral proteins, and/or analogs thereof disclosed herein wherein at least one of the viruses, viral proteins, and/or analogs thereof are immobilized to a solid support. In some embodiments, the invention relates to compositions comprising any one or combination of viruses, viral proteins, and/or analogs thereof disclosed herein are immobilized to a solid support, wherein at least one of viruses, viral proteins, and/or analogs are coated with at least one metal surface and wherein the metal surface is covalently linked to at least one or more probes. For instance, in some embodiments, the one or more probes chosen from a single stranded nucleic acid, a naturally occurring or synthetic polypeptide sequence, and an antibody. In some embodiments the viral proteins or analogs thereof are polypeptides that are subunits of a larger filamentous protein. In some embodiments, the filamentous protein is any viral protein tat is rod-shaped, including a capsid protein, that envelopes viral nucleic acid or is derived from such proteins. In some embodiments, the filamentous protein self assembles from a plurality of subunits defined by any of the amino acid sequences disclosed herein. In some embodiments, the viral protein is a polypeptide comprised of a plurality of subunits of any one or combination of amino acid sequence disclosed herein. In some embodiments, the plurality of viral proteins and/or analogs thereof comprises no more than 2130 subunits. In some embodiments, the plurality of viral proteins and/or analogs thereof comprises no more than 2120 subunits. In some embodiments, the plurality of viral proteins and/or analogs thereof comprises no more than 2110 subunits. In some embodiments, the plurality of viral proteins and/or analogs thereof comprises no more than 2100 subunits. In some embodiments, the plurality of viral proteins and/or analogs thereof comprises no more than 2090 subunits. In some embodiments, the plurality of viral proteins and/or analogs thereof comprises no more than 2080 subunits. In some embodiments, the plurality of viral proteins and/or analogs thereof comprises no more than 2070 subunits. In some embodiments, the plurality of viral proteins and/or analogs thereof comprises no more than 2060 subunits. In some embodiments, the plurality of viral proteins and/or analogs thereof comprises no more than 2050 subunits. In some embodiments, the plurality of viral proteins and/or analogs thereof comprises no more than 2040 subunits. In some embodiments, the plurality of viral proteins and/or analogs thereof comprises no more than 2030 subunits. In some embodiments, the plurality of viral proteins and/or analogs thereof comprises no more than 2020 subunits. In some embodiments, the plurality of viral proteins and/or analogs thereof comprises no more than 2010 subunits. In some embodiments, the plurality of viral proteins and/or analogs thereof comprises no more than 2000 subunits. In some embodiments, the plurality of viral proteins and/or analogs thereof comprises no more than 1950 subunits. In some embodiments, the plurality of viral proteins and/or analogs thereof comprises no more than 1900 subunits. In some embodiments, the plurality of viral proteins and/or analogs thereof comprises no more than 1850 subunits. In some embodiments, the plurality of viral proteins and/or analogs thereof comprises no more than 1800 subunits. In some embodiments, the plurality of viral proteins and/or analogs thereof comprises no more than 1750 subunits. In some embodiments, the plurality of viral proteins and/or analogs thereof comprises no more than 1700 subunits. In some embodiments, the plurality of viral proteins and/or analogs thereof comprises no more than 1650 subunits. In some embodiments, the plurality of viral proteins and/or analogs thereof comprises no more than 1600 subunits. In some embodiments, the plurality of viral proteins and/or analogs thereof comprises no more than 1550 subunits. In some embodiments, the plurality of viral proteins and/or analogs thereof comprises no more than 1500 subunits. In some embodiments, the plurality of viral proteins and/or analogs thereof comprises no more than 1450 subunits. In some embodiments, the plurality of viral proteins and/or analogs thereof comprises no more than 1400 subunits. In some embodiments, the plurality of viral proteins and/or analogs thereof comprises no more than 1350 subunits. In some embodiments, the plurality of viral proteins and/or analogs thereof comprises no more than 1300 subunits. In some embodiments, the plurality of viral proteins and/or analogs thereof comprises no more than 1250 subunits. In some embodiments, the plurality of viral proteins and/or analogs thereof comprises no more than 1200 subunits. In some embodiments, the plurality of viral proteins and/or analogs thereof comprises no more than 1150 subunits. In some embodiments, the plurality of viral proteins and/or analogs thereof comprises no more than 1100 subunits. In some embodiments, the plurality of viral proteins and/or analogs thereof comprises no more than 1050 subunits. In some embodiments, the plurality of viral proteins and/or analogs thereof comprises no more than 1000 subunits. In some embodiments, the plurality of viral proteins and/or analogs thereof comprises no more than 900 subunits. In some embodiments, the plurality of viral proteins and/or analogs thereof comprises no more than 800 subunits. In some embodiments, the plurality of viral proteins and/or analogs thereof comprises no more than 700 subunits. In some embodiments, the plurality of viral proteins and/or analogs thereof comprises no more than 600 subunits. In some embodiments, the plurality of viral proteins and/or analogs thereof comprises no more than 500 subunits. In some embodiments, the plurality of viral proteins and/or analogs thereof comprises no more than 400 subunits. In some embodiments, the plurality of viral proteins and/or analogs thereof comprises no more than 300 subunits. In some embodiments, the plurality of viral proteins and/or analogs thereof comprises no more than 200 subunits. In some embodiments, the plurality of viral proteins and/or analogs thereof comprises no more than 100 subunits. In some embodiments, the plurality of viral proteins and/or analogs thereof comprises from about 100 subunits to about 2130 subunits of the sequences provided herein. In some embodiments, the viruses and/or viral proteins and/or analogs thereof comprise a nucleic acid sequence. In some embodiments, the viruses and/or viral proteins and/or analogs thereof do comprise a nucleic acid sequence.

The invention relates to a system, sensor, device, and compositions with increased detection sensitivity. The invention also relates to a method of increasing the sensitivity of a system, sensor, device, and compositions by adhering and/or covalently attaching viruses, viral proteins or analogs thereof to at least one solid support (which in some embodiments is an electrode), such adherence or binding increases the surface area of at least one addressable area on the at least one solid support or electrode thereby increasing the signal obtained when the electrode is exposed to an electrochemically active substrate such as a target molecule, substance, or nucleic acid molecule.

Also described herein is a device or system comprising any of the arrays, polypeptides, and/or probes disclosed herein for predicting the likelihood of a subject having a psychotic disorder, the system including: a data storage unit or memory for storing one or more association values associated with a test sample obtained from the subject, wherein the association value includes quantitative expression data for the presence, absence, or quantity of polymorphisms related to the psychotic disorder in a subject.

Also described herein is a device or system comprising any of the arrays, polypeptides, and/or probes disclosed herein for diagnosing a subject with a psychotic disorder, the device or system including: a data storage unit or memory for storing one or more association values associated with a test sample obtained from the subject, wherein the association value includes quantitative expression data for the presence, absence, or quantity of polymorphisms in a subject.

Also described herein is a device or system comprising any of the arrays, polypeptides, and/or probes disclosed herein for diagnosing a subject with an infection of a pathogenic microorganism, the device or system including: a data storage unit or memory for storing one or more association values associated with a test sample obtained from the subject, wherein the association value includes quantitative expression data for the presence, absence, or quantity of a pathogenic microorganism in a subject.

Also described herein is a device or system comprising any of the arrays, polypeptides, and/or probes disclosed herein for diagnosing a subject with exposure to a contaminant, the device or system including: a data storage unit or memory for storing one or more association values associated with a test sample obtained from the subject, wherein the association value includes quantitative expression data for the presence, absence, or quantity of contaminants in a test sample obtained from the subject or from a geographic location to which the subject was exposed.

The invention also relates to a method of monitoring the amount of clozapine in a subject comprising: detecting the presence of clozapine in one or more test sample from a subject taking clozapine. In some embodiments, any or all of the steps of the methods disclosed herein may be performed using a kit described herein.

In any embodiments of methods disclosed herein, the probe used to determine the likelihood of a subject having a psychotic disorder is any ssDNA molecule or probe described herein.

The invention also relates to a method of determining patient responsiveness to a therapy for psychotic disorders comprising:

(a) contacting any of the arrays, devices, or compositions disclosed herein with the test sample comprising or thought to comprise a target molecule or substance;

(b) detecting the presence, absence or quantity of the target molecule or substance in the test sample;

(c) determining one or more association values based upon the presence, absence, or quantity of the target molecule or substance in the test sample; and

(d) predicting patient responsiveness to a therapy to treat or prevent a psychotic disorder based upon the association value; wherein the method optionally comprises the step of providing a test sample and/or control sample from subjects on clozapine and not on clozapine;

### Sensors

In some embodiments, the invention relates to a device or sensor comprising the array disclosed herein wherein the solid support comprises a surface area that is at least partially if not fully an electrode operably connected to a circuit comprising a voltmeter and/or an ammeter. In some embodiments, the device or sensor comprises a storage memory.

### Characterizing Samples and Association Values

The present invention encompasses the recognition that association signatures characteristic of particular cells of interest are useful in a variety of contexts, for example to identify, characterize, detect, and/or isolate substances or molecules of interest.

The present invention provides systems for determining association signatures characteristic of a given test sample.

The present invention relates to any of the methods described herein comprising a step of determining an association value associated with the presence, absence or quantity of a substance or target molecule. The present invention relates to a relationship that substances can be identified, detected and/or quantified by signals that are proportional to affinity between one or more disclosed components on a disclosed solid support and its binding partner or target molecule. In some embodiments, an association signature includes binding information sufficient to compare a particular quantity of interest with a reference sample and/or to identify, characterize, and/or distinguish a particular test sample with respect to other reference that do not comprise a target substance or target molecule.

In some embodiments, an association signature comprises information respecting absence, presence and/or level of binding interactions with one or more components selected from and combinations thereof.

In some embodiments, an association signature distinguishes sample from comparable samples of other tissue origin. In some embodiments, an association signature distinguishes test sample type from comparable samples of a different developmental stage (or point in development). In some embodiments, an association signature distinguishes a sample from comparable sample that differ in presence of and/or susceptibility to one or more disease states, disorders, or conditions. In some embodiments, an association signature distinguishes a test sample from comparable sample taken from a similar geographically placed water resource. In some embodiments, an association signature distinguishes a test sample from comparable sample taken from a similar geographically placed food source. In some embodiments, an association signature distinguishes a test sample from comparable reference sample that differs with respect to extent, degree, or type of exposure to one or more environmental factors (including drugs, toxins, contaminants, etc).

In some embodiments, detection or determination of an association signature reveals information about identity, extent, and or nature of one or more components contained within a test sample, and/or of one or more factors present on (e.g., expressed or captured on) in a test sample or on a surface. To give but one example, existence and/or level of particular binding interactions in an association signature of a sample can reveal identity, extent, and or nature of a sample or surface component such as, for example, a contaminant binding a probe specific to the contaminant. Presence of the contaminant can indicate a hazardous condition or place that should be avoided or cleaned prior to use, ingestion or exposure by a subject.

In some embodiments, association signatures are determined by contacting a sample with an array or system disclosed herein; quantifying one or more association values; and compiling the one or more association values to create one or more association signatures. In some embodiments, the step of quantifying one or more association values comprises detecting a quantitative signal or signals relative to the sample binding to one or a plurality of association probes, normalizing the quantitative signals as compared to a control sample, and applying an algorithm or interpretation function disclosed herein to the quantitative signal or signals such that the output of the algorithm or interpretation function disclosed herein comprises one or a plurality of association values. In some embodiments, the step of applying the algorithm or interpretation function disclosed herein is performed by a non-transitory computer program product. In some embodiments, one or more steps of the methods disclosed herein are performed by a non-transitory computer implemented method. In some embodiments, association signatures or association values are one or a collection of ammeter or voltmeter readings related to the presence or quantity of electrochemical agent substance or target molecule bound to any of the probes disclosed herein and detected by the one or more electrodes disclosed herein.

Once the one or plurality of association values are calculated using the algorithm or interpretation function, one can create or determine an association signature for the sample which, in some embodiments, is a quantitative binding profile of sample relative to the one or plurality of modified viruses or viral proteins or analogs thereof to which sample has been contacted. A user of the array or system disclosed herein can subsequently compare the association signature sample to one or a plurality of association signatures of control samples. In some embodiments, the association signatures of the one or plurality of control samples is predetermined and/or catalogued so that the user of the array or system disclosed herein can compare the signatures of the sample to the predetermined and/or catalogued control signature to identify the level of molecule or contaminant in a food, water, or bodily fluid sample. In some embodiments, the association signatures of the one or plurality of controls is predetermined and/or catalogued so that the user of the array or system disclosed herein can compare the signatures of the test sample to the predetermined and/or catalogued control association signature to qualitatively assess the test sample as having physical characteristics more or less similar to the control association signature. In some embodiments, the user of the array or system disclosed herein and generate a profile related to similarities or dissimilarities as between the test sample association signature and the control association signature. In some embodiments, the control association signature is association signature that quantitatively describes a set of association values from a panel of bodily fluid. In some embodiments, the control association signature is an association signature that quantitatively describes a set of association values from nervous tissue. In some embodiments, the control association signature is association signature that quantitatively describes a set of association values from bodily fluid or tissue taken form a subject being diagnosed with or suspected of having a psychotic disorder. In some embodiments, the control association signature is association signature that quantitatively describes a set of association values from a cell. In some embodiments, the control association signature is association signature that quantitatively describes a set of association values from an embryonic stem cell. In some embodiments, the control association signature is association signature that quantitatively describes a set of association values from a mesenchymal stem cell. In some embodiments, the control association signature is association signature that quantitatively describes a set of association values from an induced pluripotent stem cell. In some embodiments, the control association signature is association signature that quantitatively describes a set of association values from one or various degrees of a mental disorder.

In general, one challenge faced by researchers and medical professionals is a need to identify cell types, differentiation states, and phenotypes, and to adequately isolate and grow specific cell populations. For example, because of interplay between genetic and environmental factors, two sub-populations of cells may be genetically identical and differ detectably only in composition of their components. Thus, one advantage of determining association signature of cells as provided herein is that it can permit researchers to distinguish between cell populations that have not previously been distinguishable. Alternatively or additionally, provided methods and compositions allow characterization and/or classification of test samples in ways not previously available or appreciated. Provided methods and compositions also provide basis for isolation or separation of target molecules and substances from one another and/or from other components, materials, or entities. Any association values may be a voltage reading or amperage reading detected by the presence of the target molecules or substance bound to the one or more probes in the disclosed device, sensors, compositions, or arrays. Any association signatures may include the relation between the current peak and the surface area of the electrode of the sensor, array, or device described herein. Such correlation may be determined by the methods outlined in Electrochemical Methods, Fundamentals and Applications, Bard and Faulkner Wiley, (2001), pages 1-850, especially but not limited to the equations of page 231. This book is incorporated by reference in its entirety.

The invention relates to methods of diagnosing a subject with a psychotic disorder, the method comprising detecting the presence, absence, or quantity of a target molecule or substance in a sample from the subject using any of the devices, arrays, sensors or compositions disclosed herein.

In some embodiments, any of the devices, sensors, or compositions disclosed herein comprise one or a plurality of viruses or viral proteins covalently bound to a probe. In some embodiments, any of the devices, sensors, or compositions disclosed herein comprise one or a plurality of layers of gold covalently bound to a probe. In some embodiments, any of the devices, sensors, or compositions disclosed herein comprise one or more DNA probe. In some embodiments, cell lysates are probed with DNA probes that allow cells with different characteristics to be distinguished by genotype or phenotype. In some embodiments, cells are probed with DNA probes that allow cells with different characteristics to be distinguished by RNA transcript expression number. In some embodiments, any of the devices, sensors, or compositions disclosed herein comprise one or more cofactors. In some embodiments, any of the devices, sensors, or compositions disclosed herein comprise probes that are enzymes or substrates that allow characteristics to be distinguished by enzymatic activity. In some embodiments, cells are probed with an antibody that allow cells with different characteristics to be distinguished by affinity for proteins.

### Diagnosis

As described above, certain embodiments of the present invention may be used to distinguish between samples at different states of infection, making it a promising tool for diagnosing infection by a pathogenic microorganism. This system is potentially useful, for example, when testing samples of a patient to determine whether disease is present. Diagnosing a patient using association signatures would include, for example, comparing an association signature of a sample from a patient with and association signature of reference cells.

In certain embodiments, association signatures are used to diagnose and/or prognose a patient suspected of having any condition causing his or her cells to have a distinguishing characteristic from reference sample as a result of the condition. In certain embodiments, association signatures are used to diagnose and/or prognose a patient suspected of having any disease that affects association signatures. In certain embodiments, association signatures are used to diagnose and/or prognose a patient suspected of having any form of psychotic disorder. In certain embodiments, association signatures are used to diagnose and/or prognose a patient suspected of having schizophrenia. In certain embodiments, association signatures are used to diagnose and/or prognose a patient suspected of having psychosocial disorder. In certain embodiments, association signatures are used to diagnose and/or prognose a patient suspected of having a psychotic disorder.

In some embodiments, kits in accordance with the disclosure provide a means of diagnosing an infection with a microorganism disclosed herein. The sensors and devices and compositions herein provide tools for diagnosis and/or prognosis via association signatures in kit form.

In some embodiments, presence of nucleic acids from pathogenic microorganisms is detected by affinity for a probe with the nucleic acids from the pathogenic microorganisms. In some embodiments, the kit further comprises a means for assessing growth or abundance of the pathogenic microorganisms. Methods for detecting and/or assessing cell growth and/or abundance are well known in the art and include but are not limited to spectrophotometry, FACS, microscopy, and/or plating. In some embodiments, a means for assessing growth or abundance of the pathogenic microorganisms comprises a container for sending the kit to a facility where growth and/or abundance is assessed before or after detection methods are performed.

### Protein or Substrate Isolation

In some embodiments, methods in accordance with the disclosure may be used as a tool to isolate proteins or a substance (such as a contaminant) of interest. This system is useful, for example, when trying to isolate certain types of polypeptides or nucleic acids out of a mixed tissue or cell population. When given a complex mixture of cells, for example partially differentiated stem cells, a patient biopsy, or a bone marrow sample, deconvolving this mixture using traditional methods can be difficult. In general, it is thought that one of the easiest ways to achieve this result is by flow cytometry, but flow cytometry requires an initial prediction of what might be present in a sample to establish a panel of markers that would represent that population. In some embodiments of the present invention, the use of association signatures simplifies this process.

In many embodiments, an array comprises a solid support to whose surface(s) viral components are affixed in spatially discrete locations. Such an array can be prepared using viral components from any source (e.g., recombinantly produced, biochemically isolated, commercially purchased, etc). Moreover, identity and relative amounts of individual viral components may be determined or adjusted in accordance with requirements of a particular project or interests of a particular researcher.

For example, in many embodiments, it will be desirable to design, prepare and/or utilize an array that includes as many different components as is feasible. Alternatively or additionally, in some embodiments, it may be desirable to design, prepare, and/or utilize an array that includes only viral components known to be associated with (or not associated with) a particular shape and/or dimension so as to provide any or all of the disclosed devices, sensors, systems or compositions with as much surface area for sensing as possible on an electrode. To give a few particular examples, in some embodiments, an array is utilized that contains at least 1, 2, 3, 4, 5, 6, 7, 8, 9, 10, 11, 12, 13, 14, 15, 16, 17, 18, 19, 20 or more different “spots” (physically discrete locations) containing different viral components. In some embodiments, an array is utilized that contains from about 1 to about 1000,000 viruses, from about 100 and about 1×10⁶ viruses, or between about 1,000 and about 1×10⁷ viruses. The TMV dimensions are 300 nm in length and 18 nm in diameter. Under ideal conditions the surface area of the electrode that is occupied by a single TMV is πr², hence 2.55*10⁻¹⁰ mm². For example, a square planar electrode with surface area of 49 mm² will bind 1.93*10¹¹ viruses. Therefore, the calculated maximum virus density bound to an electrode is 3.93*10⁹ virus/mm²

In some embodiments, discrete addressable locations on an array show spatial organization. In some embodiments, the viruses, viral proteins, or analogs thereof are arranged in a grid or a fully confluent layer on the solid support.

In some embodiments, a variety of viral components and combinations thereof are represented on addressable location of the array with each addressable location corresponding to both a known location on the array and a known composition of viruses or viral components or modification of such viruses or viral components. In certain embodiments, at least one virus or viral component is spotted upon the array. In certain embodiments, the one virus or viral components are spotted individually. In some embodiments, mixtures of several viruses or viral components are contained within a single spot. In some embodiments, an array for use in accordance with the present invention includes both spots of single viruses or viral components and spots of combinations of viruses and viral components. In some embodiments, one virus or viral components are spotted multiple times in the same array, so that the array includes replicate spots. In some embodiments, an array for use in accordance with the present invention contains spots that lack a virus or viral component with a modification or linker or probe, and therefore, for example, may be utilized as negative controls in addition to spots containing one or a plurality of virus or viral components.

Deposition of metal may be applied to the solid support (in some embodiments made of silica), for

After deposition of metal layers, arrays may be made by covalently bonding any thiol-containing chemical group to the surface of the gold using methods outlined in Analyst, April 1997, Vol. 122 (43R-50R), incorporated herein by reference in its entirety.

An array for use in accordance with the present invention may be prepared on any suitable substrate material. In many embodiments, the material will support electroconductivity and/or will comprise an operable linkage to one or more voltmeters or ammeters. In some embodiments, the arrays, sensors, devices and or compositions disclosed herein utilizes a substrate material selected from the group consisting of polyamides, polyesters, polystyrene, polypropylene, polyacrylates, polyvinyl compounds (e.g. polyvinylchloride), polycarbonate, polytetrafluoroethylene (PTFE), nitrocellulose, cotton, polyglycolic acid (PGA), cellulose, dextran, gelatin, glass, fluoropolymers, fluorinated ethylene propylene, polyvinylidene, polydimethylsiloxane, polystyrene, silicon substrates (such as fused silica, polysilicon, or single silicon crystals), and the like, or combinations thereof. Alternatively or additionally, metals (gold, silver, titanium films) can be used on one or more surfaces of the substrate. In a some embodiments, the substrate material comprises an electroconductive surface operably linked to one or more voltmeters or amperometers.

In some embodiments, the present invention provides arrays for use as diagnostic assays for the diagnosis of psychosocial disorder, schizophrenia, and psychotic pathology. In some embodiments the arrays are provided as part of a diagnostic or detection kit for the likelihood of having or being diagnosed with psychosocial disorder, schizophrenia, and psychotic pathology. In some embodiments the arrays are provided as part of a detection kit. In certain embodiments, kits for use in accordance with the present invention may include one or more reference samples, non-specific probes (non-specific as to the target molecule of interest); instructions (e.g., for processing samples, for performing tests, for interpreting results, etc.); media; and/or other reagents necessary for performing tests.

Various modifications, adaptations, and combinations of various features of the described embodiments can be practiced without departing from the scope of the invention as set forth in the claims. Any and all journal articles, patent applications, issued patents, or other cited references are incorporated by reference in their entirety.
1. “Carbon nanotube/teflon composite electrochemical sensors and biosensors,” J. Wang, and M. Musameh, Analytical Chemistry, 75, 2075 (2003).
2. “Nanostructured nickel electrodes using the Tobacco Mosaic Virus for microbattery applications,” K. Gerasopoulos, M. McCarthy, E. Royston, J. N, Culver, and R. Ghodssi, Journal of Micromechanics and Microengineering, 18, 104003 (2008).
3. “Biofabrication methods for the patterned assembly and synthesis of viral nanotemplates,” K. Gerasopoulos, M. McCarthy, P. Banerjee, X. Fan, J. N. Culver, and R. Ghodssi, Nanotechnology, 21, 055304 (2010).
4. “Neuregulin signaling, cortical circuitry development and schizophrenia,” B. Rico, and 0. Marin, Current Opinion in Genetics & Development, 21, 262 (2011).
5. “Probing biomolecular interactions at conductive and semiconductive surfaces by impedance spectroscopy: routes to impedimetric immunosensors, DNA-sensors, and enzyme biosensors,” E. Katz, and I. Willner, Electroanalysis, 15, 913 (2003).

### EXAMPLES

### Example 1

### Array Manufacture

The following example describes an array. Among other things, the present invention provides a collection of viruses or viral proteins attached to a solid surface useful in accordance with the present invention to define, detect, or utilize one or more association values assigned to target sample or characterize a target surface.

In some embodiments electrochemical sensors were fabricated by aligning rod-shaped 300×18 nm TMV vertical viruses [2, 3] on microfabricated planar square gold electrodes (Surface area=0.49 cm²) via genetically engineered coat protein cysteine. TMV2cys was created using a polymerase chain reaction (PCR)-based mutagenesis technique. The design of this mutation resulted in the insertion of two cysteine residues at positions 2 and 3 within the TMV coat protein open reading frame (ORF). PCR amplification of the mutant coat protein ORF was performed using a 5′ coat protein specific DNA primer that contained a BglII restriction site as well as the additional cysteine codons and a 3′ coat protein specific primer containing a BsiWI restriction site. The mutant coat protein gene was then cloned into a full-length infectious cDNA clone of TMV using the above primer incorporated restriction sites to create TMV2cys. Infectious RNA transcripts of the modified virus were generated and used to inoculate leaves of Nicotiana tabacum cv Xanthi. TMV2cys virions were extracted from infected leaves and purified on a 10-40% sucrose gradient and suspended in 0.01 M phosphate buffer. Sequence analysis of cDNA derived from purified virions demonstrated that the 2cys mutation was maintained within the viral genome. (from 2005 Lee et al. Nanotechnology)

The TMV genome encodes a rod-shaped polypeptide particle 300 nm in length and 18 nm in diameter with a 4 nm diameter hollow inner channel. Each TMV particle is composed of ˜2130 identical protein subunits of molecular weight 17.5 kDa that self-assemble in a helix around a single strand of genomic virus RNA.

A full-length infectious cDNA clone of the U1 strain of TMV, pSNC004, was used as the parental construct for the creation of TMV 1 cys. The addition of a single cysteine residue within the TMV coat protein was created by the insertion of a TGT codon at the third position within the coat protein open reading frame using a PCR-based mutagenesis procedure. Infectious RNA transcripts generated from the full-length TMV1cys construct were used to inoculate Nicotiana tabacum, cv Xanthi, a systemic TMV host. Inoculated plants were harvested at 20 days postinoculation and virus purified as previously described. (Yi H, Nisar S, Lee S Y, Powers M A, Bentley W E, Payne G F, Ghodssi R, Rubloff G W, Harris M T, Culver J N. 2005. Patterned assembly of genetically modified viral nanotemplates via nucleic acid hybridization. Nano Lett. October, 5(10): 1931-6.)

Electrodes were fabricated with a microfabrication technology. SiO₂ was grown on a silicon wafer (single side polished) through a PECVD process. Then 20 nm chrome layer was sputtered on the Silicon/SiO₂ wafer, followed by 180 nm of gold layer. The electrodes were patterned on the chrome/gold coated wafers through a photolithography process. A thin layer of positive photoresist (“S1813”; ˜15 micron) was spun on top of the coated wafer, followed by pre-exposure step of baking at 100 celsius for 1 minute. The baked wafer was exposed with a mask aligner tool for 12 seconds to pattern the electrodes. Following exposure, electrode patterns were developed in a photoresist developer (“352 developer”) for 30 seconds. Then the photoresist was stripped with acetone and the wafer was diced into single electrode chips with an electrode dimensions of 7 mm×7 mm. The single electrode chips were incubated overnight (˜18 hours) with a solution of TMV-1cys nano-particles suspended in phosphate buffer (pH=7). The TMV bound electrodes were activated with a solution of sodium tetrachloropalladate for 3 hours. The palladium activated TMV was then incubated in a nickel electroless plating solution for 5 minutes, coating the TMV outer surface with nickel layer. Then, the nickel coated TMV was incubated in a gold plating bath for 45 minutes at 90 celsius

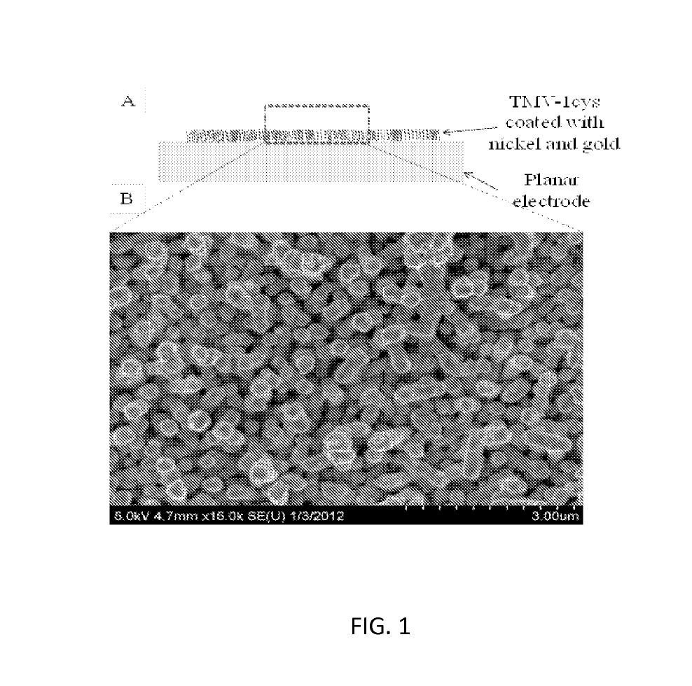

FIG. 1 shows SEM images of the surface of the electrode modified with nickel-coated TMV-1cys after gold deposition. TMV-1cys were observed to attach vertically on the electrode surface. A TEM image of the nanocomposite virus particle (FIG. 2) shows approximately 67 nm nickel and 46 nm gold coatings. The thin films of the nickel and the gold maintain the high aspect ratio of the TMV scaffold increasing the electrochemically active surface area of the electrode.

### Example 2

### Array Fabrication and Detection

In the following example, an expanded array is developed for the purpose of identifying the presence of a polymorphism in the neuregulin gene (NRG1). Mental health analysis was used as a model sensing system, when the target neuregulin-1 (NRG 1) polymorphism A single nucleotide polymorphism of the neuregulin 1 gene (SNP8NRG243177/rs6994992) increases the risk of psychosis, affects prefrontal activation and structural connectivity in the brain, and is related to the expression of a specific neuregulin 1 isoform. Patients with the risk T/T genotype express more unusual thoughts than C-carriers (C/T and C/C) during conflict-related interactions but not during neutral interactions. There is a significant gene by environment interaction regarding SNP8NRG243177/rs6994992 and psychosocial stress (Keri, S., Kiss, I., Seres, I. and Kelemen, O. (2009), Am. J. Med. Genet., 150B: 418-420).

If the presence of the polymorphism is detected, one of ordinary skill can correlate its presence to a diagnosis to psychosocial disorders and psychosis. If the presence of the polymorphism is detected, one of ordinary skill can correlate its presence to an increased likelihood of developing or having psychosocial disorder or psychosis. A single-stranded DNA (ssDNA) was synthesized as defined below. ssDNA probe sequence: 5′-TA CAT TCA CTT GAA CCC TGC ATG GTG CTT C-3′ thiolated in the 5 prime) (SEQ ID NO:8). Complementary target ssDNA (SNP8NRG243177 polymorphism): 5′-GAA GCA CCA TGC AGG GTT CAA GTG AAT GTA-3′ (SEQ ID NO:9). Non-complementary ssDNA: 5′-CTT CGT GGT ACG TCC CAA GTT CAC TTA CAT-3′ (SEQ ID NO:10). The TMV/Ni/Au modified electrode was incubated in the probe solution for 1 hour followed by incubation in 6-mercapto-1-hexanol chemical solution for 3 hours to prevent non-specific adsorption. The modified electrode functionalized with the ssDNA probe was introduced to a ferrocyanide/ferricyanide redox couple solution and cyclic voltammetry was measured with electrochemical setup, a commercial Ag/AgCl reference electrode and a platinum wire as the counter electrode. Then the functionalized electrode was incubated for 20 minutes to either a non-complementary ssDNA or a complementary target ssDNA, and cyclic voltammetry was measured in the presence of a ferrocyanide/ferricyanide redox couple solution following each incubation with electrochemical setup, a commercial Ag/AgCl reference electrode and a platinum wire as the counter electrode.

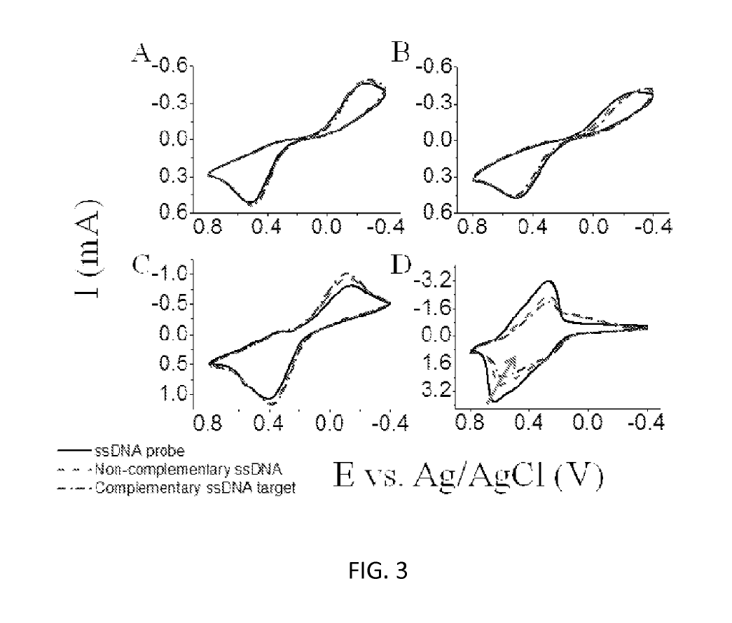

ssDNA (SNP8NRG243177) detection efficiency was electrochemically analyzed using thiolated ssDNA probe encoding human NRG1 assembled on the TMV/Ni/Au modified electrode. Open circuit potential analysis of the introduced noncomplementary and complementary NRG 1 target ssDNAs demonstrated potential reduction when DNA hybridization event occurred. This reduction is due to an increase with the total negative charge at the surface of the electrode when hybridization event of two negatively charged ssDNA occurs. The modified electrodes were electrochemically characterized using cyclic voltammetry in the presence of a redox couple ferrocyanide/ferricyanide solution. FIG. 3 highlights the increased signal from the modified electrodes with cyclic voltammograms of the generated electrochemical reduction and oxidation reactions. The electrodes produced reversible nernstian characteristics where the TMV/Ni—Au modified electrode generated the highest electrochemical current (8 fold higher oxidation peak current in comparison with the unmodified planar electrode) due to the high active surface area. Effective surface area calculations demonstrated that TMV/Ni/Au resulted in the highest area (Table 1), a characteristic important for the high performance of electrochemical sensors.

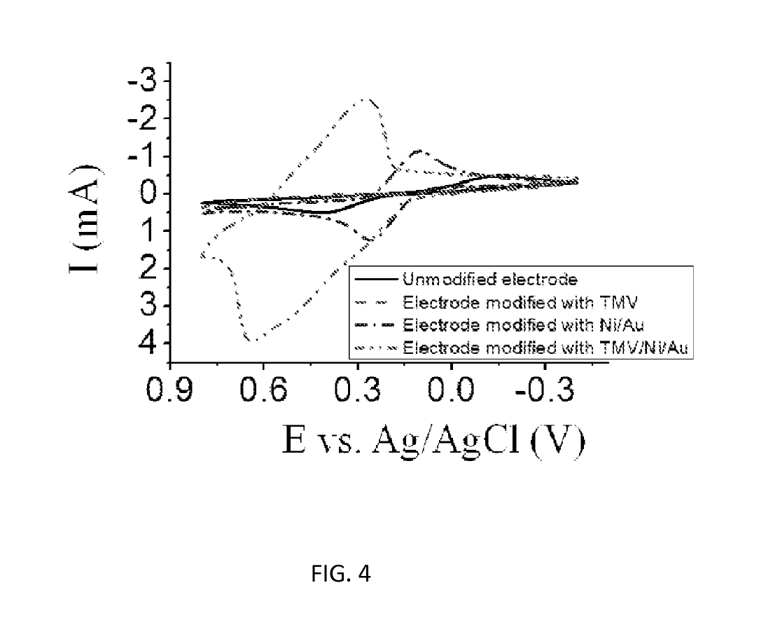

Sensing performance was studied with cyclic voltammetry (FIG. 4). All electrodes demonstrated reversible nernstian characteristics in the presence of complementary NRG1 target and non-complementary ssDNA. Moreover, TMV/Ni/Au modified electrode demonstrated the largest current differentiation upon DNA hybridization. DNA hybridization causes stronger electrostatic repulsion forces with the negatively charged electro-active species in the electrolyte, hence impeding the electrochemical reaction at the electrode [5]. Detection efficiency (oxidation peak current decrease between noncomplementary and complementary ssDNA) analysis (Table 1) demonstrated 9.5 fold improved biosensing performance for the 3D TMV/Ni/Au modified electrode. Peak current was detected by the following equation:

$${I}_{peak} = 0.4463 ( \frac{F}{R T} ) {n}^{3 / 2} A {D}^{1 / 2} {C}^{*} {v}^{1 / 2}$$

F [C mol⁻¹]—Faraday constant
R [J mol⁻¹ K⁻¹]—gas constant
T [K]—temperature
n—stoichiometric number of electrons involved in an electrode reaction
A [cm²]—effective surface area of the electrode
D [cm² s⁻]—diffusion coefficient of the electro-active species
C* [mol cm⁻³]—bulk concentration of the electro-active species
v [V s⁻¹]—linear potential scan rate
Iₚₑₐₖ [A]—peak current

Evaluation of the sensitivity, detection range, and limit of detection will be performed by introducing different concentrations of the target analyte and measuring the resulted electrochemical signal. The resultant signals will be used to construct a dose response plot that can be used to correlate the signal to the corresponding target concentration. The target concentration can be correlated to the quantity and the mass through the molecular weight of the target. For example, for the DNA hybridization sensor, different concentrations of a target ssDNA will be introduced to the TMV-modified electrode and the resulted signal will be measured. Then, the measured cyclic voltammetry signals will be correlated to the ssDNA target concentration through a dose response plot. As the electron transfer resistance increases for increased DNA hybridization events, the cyclic voltammetry signal will decrease for higher number of hybridization events, hence higher concentration of the target ssDNA. The anticipated correlation plot will have a negative relationship with a linear region that will be used to correlate the measured ssDNA target concentration.

This work demonstrates the first utilization of virus molecules as a nano-scale biotemplates assembled on an electrochemical sensor, allowing for an increased signal of a magnitude of 8 and an improved sensing performance of 9.5 fold. The integration of versatile and inexpensive biological Tobacco Mosaic Virus (TMV) as a high aspect ratio, low foot print, low cost, easy to genetically functionalized, nanostructured three-dimensional scaffold for the synthesis of novel multifunctional electrodes with an increased surface area resulting in higher electrochemical currents, better signal-to-noise ratio and improved sensitivity when incorporated into sensors. Table 1 below illustrates the measure of biodetection efficiency analysis for modified and unmodified electrodes. The biodetection efficiency was calculated as follows: [(Current at the peak measured with a non-complememntray ssDNA)−(Current at the peak measured with a target complememntray ssDNA)]/(Current at the peak measured with a non-complememntray ssDNA).

TABLE 1

| Electrode | Effective surface | Detection |
|---|---|---|
| modification | area [cm²] | efficiency [%] |
| Unmodified | 0.42 | 1.7 |
| TMV | 0.32 | 2.1 |
| Ni/Au | 1.05 | −1.1 |
| TMV/Ni/Au | 3.34 | 16.1 |

### Example 3

### Using the Array to Assay Food Safety

Following fabrication of the TMV/Ni/Au modified electrode, the surface of the modified electrode will be incubated with antibodies for parathion through a linker-thiol binding reaction. The sensitivity, limit of detection, and detection range of the sensor will be evaluated by incubating the biosensor with different concentrations of parathion and measuring the electrochemical activity in the presence of ferrocyanide/ferricyanide redox couple. The redox couple will be detected with electrochemical methods such as cyclic voltammetry, chrono-coulometry, differential pulse voltammetry, and electrochemical impedance spectroscopy, but not only these. The sample amounts varies between 10 microliter to 1 milliliter and higher. For dose-response samples of parathion either in phosphate buffer saline or water from the environment in concentrations between 1 fM and 1 mM will be tested with the sensor and the corresponding oxidation signal at the peak of the cyclic voltammetry or the DPV techniques, or the total oxidative charge measured with chrono-coulometry, or the change with the charge transfer resistance with electrochemical impedance spectroscopy, will be measured and plotted in relationship to the parathion concentration. The expected relationship would be a negative relation as higher concentration of parathion in the sample would result in higher number of antibody-antigen attachment reactions, hence decrease the exposed area of the electrode and decrease the oxidation current. Following dose-response and correlation analysis, the antibody-functionalized electrodes will be introduced to grounded food and will be tested for the presence of parathion. The food samples will be taken from different locations of the tested environment and will be correlated to measurements with commercial testing in the laboratory. Following dose-response and correlation analysis, the antibody-functionalized electrodes will be introduced to grounded food and will be tested for the presence of parathion.

In both this example and Example 4, one or more probes specific for pathogenicity of microorganisms may be used to detect the presence of pathogenic microorganism in a test sample. The probes may be single stranded nucleic acids that are complementary to

1. Escherichia coli O157:H7—eaeA gene
2. Bacillus anthracis—protective antigen A (pagA) gene (accession number, M22589—incorporated by reference in its entirety);
3. insertion element (Iel) gene (accession number, Z83734 incorporated by reference in its entirety) of Salmonella enteritidis
4. Vibrio cholerae—cholera toxin (CT) genes, ctxAB.

One of ordinary skill in the art can design probes complementary to the sequences disclosed above. In some embodiments, the probes are from about 10 base pairs to about 40 base pairs of nucleotides complementary to any contiguous sequence identified within the above-identified sequences. methods of identifying the pathogenicity islands and making probes are identified in each of: Nature, Vol 406, 3 Aug. 2000, pages 477-484; D. Zhang et al., Biosensors and Bioelectronics 26 (2010) 1736-1742; and X. Mao et al., Biosensors and Bioelectronics 21 (2006) 1178-1185, each of which is incorporated by reference in its entirety.

### Example 4

### Using the Array to Identify Water Contaminants

Following fabrication of the TMV/Ni/Au modified electrode, the surface of the modified electrode will be incubated with antibodies for the carcinogenous chemical 2-amino-3-methylimidazo[4,5-f]quinoline (IQ) through a linker-thiol binding reaction. The sensitivity, limit of detection, and detection range of the sensor will be evaluated by incubating the biosensor with different concentrations of IQ and measuring the electrochemical activity in the presence of ferrocyanide/ferricyanide redox couple. The redox couple will be detected with electrochemical methods such as cyclic voltammetry, chrono-coulometry, differential pulse voltammetry, and electrochemical impedance spectroscopy, but not only these. The sample amounts varies between 10 microliter to 1 milliliter and higher. For dose-response samples of IQ either in phosphate buffer saline or water from the environment in concentrations between 1 fM and 1 mM will be tested with the sensor and the corresponding oxidation signal at the peak of the cyclic voltammetry or the DPV techniques, or the total oxidative charge measured with chrono-coulometry, or the change with the charge transfer resistance with electrochemical impedance spectroscopy, will be measured and plotted in relationship to the IQ concentration. The expected relationship would be a negative relation as higher concentration of IQ in the sample would result in higher number of antibody-antigen attachment reactions, hence decrease the exposed area of the electrode and decrease the oxidation current. Following dose-response and correlation analysis, the antibody-functionalized electrodes will be introduced to IQ-contaminated water and will be tested for the presence of IQ. The water samples will be taken from different locations of the tested environment and will be correlated to measurements with commercial testing in the laboratory.

### Example 5

### Using the Array to Screen Drugs

Despite the risk of agranulocytosis, metabolic side effects, and a variety of cardiovascular problems, including myocarditis, the specific advantages of clozapine with regard to efficacy can still make it a prudent choice for as many as 30-40% of patients with schizophrenia or schizoaffective disorder. The following advantages are well established: 1) superiority for positive symptoms in treatment-resistant patients; 2) lower risk for suicide; 3) lower risk for tardive dyskinesia and suppression of established tardive dyskinesia; 4) improvement in cognition contributing to better work and social function; 5) higher quality of life and longer time to discontinuation; and, 6) decreased relapse. On the other hand, there are serious risks: 1) agranulocytosis; 2) insulin resistance with increased risk of type II diabetes, weight gain, and various vascular complications; and, 3) myocarditis. There are also other unpleasant side effects, such as hypersalivation and increased risk of seizures. The results from the U.S. and Finnish epidemiologic studies suggest that none of the potentially life threatening risks, as managed in the “real world,” have enough of an impact to negate the advantages which come from clozapine. However, clozapine has been available for twenty years in the U.S. now, and there is no evidence that mortality in the clozapine patients who have been taking it for this extended period is increasing. The success of clozapine in terms of time to discontinuation and fewer relapses than almost any other drug suggests that once patients get started on clozapine, their subjective experience is sufficiently favorable that they prefer it to other treatments. We will fabricate a sensor to detect levels of clozapine in the blood serum in order to minimize the likelihood of experiencing adverse side effects in a subject.

Following fabrication of the TMV/Ni/Au modified electrode as previously described above we will modify the gold surface of the sensor with an antibody (probe) that binds to clozapine. In some embodiments, any antibody that binds clozapine may be used including but not limited to the antibodies described in US Application No. 2012/0301973, which is incorporated by reference in its entirety.

Following fabrication of the TMV/Ni/Au modified electrode, the sensitivity, limit of detection, and detection range of the sensor will be evaluated by incubating the biosensor with different concentrations of the antipsychotic drug clozapine. As clozapine is an electro-active analyte, its corresponding oxidation signal will be positively correlated with increasing concentration. The clozapine will be detected with electrochemical methods such as cyclic voltammetry, chrono-coulometry, differential pulse voltammetry, and electrochemical impedance spectroscopy, but not only these. The sample amounts varies between 10 microliter to 1 milliliter and higher. For dose-response samples of clozapine spiked in either phosphate buffer saline or human serum in concentrations between 100 ng/ml and 1000 ng/ml will be tested with the sensor and the corresponding oxidation signal will be measured and plotted in relationship to the clozapine concentration. This plot will be used to correlate the corresponding clozapine concentration in the sample to the measured signal. The expected relationship would be a positive relation as higher concentration of clozapine in the sample would result in higher oxidation current. Following dose-response and correlation analysis, the sensing performance of the sensor to detect clozapine levels in schizophrenia patients will be evaluated. Samples will be drawn from schizophrenia patients under clozapine medication in either draw into a tube or gathering a blood draw from pricking a finger. The blood sample in a tube will be centrifugated and the serum will be tested with the sensor. For blood drop from finger pricking, the serum will be separated from the cells by using a micro-device for serum-cell separation. With these accomplishments, the biosensor will be used to quantify clozapine blood levels in schizophrenia patients that are under clozapine treatment.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. An array of viruses or viral polypeptides, the array comprising: a solid support and a one or a combination of: (i) a plurality of viruses; (ii) a plurality of viral proteins; and/or (iii) analogs thereof, wherein at least a portion of the plurality of viruses, viral proteins and/or analogs thereof are coated by at least one metal and wherein the one or combination of (i) a plurality of viruses; (ii) a plurality of viral proteins; and/or (iii) analogs thereof are immobilized to the solid support at least one addressable location of the array.

2. The array of claim 1, wherein the solid support is an electroconductive surface optionally coated with a polymer.

3. The array of claim 1, wherein at least one of the plurality of viruses, viral proteins or analogs thereof are derived from Virgaviridae or are polypeptides from Virgaviridae and, if the viruses, viral proteins or analogs form a filamentous particle, the particle is vertically oriented on the solid support.

4. The array of claim 1, wherein the (i) a plurality of viruses; (ii) a plurality of viral proteins; and/or (iii) analogs thereof are coated with at least two layers of metals in an area of the array, wherein the layers are from about 40 to about 60 nm in depth.

5. The array of claim 1, wherein a portion of one of a combination of (i) a plurality of viruses; (ii) a plurality of viral proteins; and/or (iii) analogs thereof are coated by a layer of nickel from about 40 to about 60 nm in depth and a layer of gold from about 40 to about 60 nm in depth.

6. The array of claim 1, wherein, if the array comprises at least one or a plurality of viruses, the viruses are Tobacco Mosaic Virus (TMV) and wherein, if the array comprises viral polypeptides or analogs of the same, the viral polypeptides are TMV capsid protein or analogs thereof with at least 70% sequence identity to SEQ ID NO: 1.

7. The array of claim 4, wherein the solid support comprises from about 2.0 to about 3.5 square centimeters of addressable positions on the array.

8. The array of claim 1, wherein the virus, viral polypeptides, or analogs thereof are modified with at least one probe and, optionally, with a linker covalently bonded to the probe.

9. The array of claim 1, wherein the array comprises a biodetection efficiency from about 2 to about 8 times the biodetetction efficiency of an array that does not comprise the one or a combination of: (i) a plurality of viruses; (ii) a plurality of viral proteins; and/or (iii) analogs thereof, wherein at least a portion of the plurality of viruses, viral proteins and/or analogs thereof.

10. The array of claim 1, wherein the solid support is planar or substantially planar surface comprising at least one electroconductive material.

11. The array of claim 7, wherein the probe is an antibody or single stranded nucleic acid.

12. The array of claim 1 configured for portable use in a sensor.

13. The array of claim 3, wherein the detection efficiency of the array is at least five times as great as the detection efficiency of an array that does not comprise viruses or viral polypeptides vertically oriented on a solid support.

14. The array of claim 1 further comprising a target molecule or substance immobilized to a probe covalently linked to the at least one metal or the one or combination of virus, viral polypeptides, or analogs thereof.

15. The array of claim 1 operably connected to at least one voltmeter or ammeter.

16. A sensor comprising the array of claim 1 configured for detection of one or more substances or molecules and comprising at least one voltmeter or ammeter in operable connection to the solid support.

17. The sensor of 16, wherein the solid support comprises at least a first and a second layer of material, the first layer of material comprising an electroconductive material to which the viruses an/or viral polypeptides are immobilized and a second layer comprising an electroconductive material that possesses oxidation and reduction potential.

18. The sensor of claim 16 further comprising at least one reservoir in fluid communication with the solid support, wherein the reservoir comprises at least one aqueous buffer.

19. The sensor of claim 16 wherein, if the array comprises at least one or a plurality of viruses, the viruses are Tobacco Mosaic Virus (TMV) and wherein, if the array comprises viral polypeptides or analogs of the same, the viral polypeptides are TMV capsid protein or analogs thereof with at least 70% sequence identity to SEQ ID NO:1.

20. The sensor of claim 16, wherein the virus, viral polypeptides, or analogs thereof are modified with at least one probe and, optionally, with a linker covalently bonded to the probe.

21. A kit comprising the array of claim 1.

22. The kit of claim 21 further comprising at least one of the following: a swab, element, a volume of fluorescent stain or dye, a volume of preservation solution, and a set of instructions, optionally accessible remotely through an electronic medium.

23. A method of identifying an association value of a sample comprising: contacting the sample to the array of claim 1; and determining a quantity of target molecule or substance bound to the array.

24. The method of claim 23, wherein the sample contains nucleic acids from a cell from a biopsy.

25. A method of diagnosing a disease or disorder or severity of a disease or disorder of a subject comprising:
(a) contacting a test sample from the subject to the array of claim 1;
(b) quantifying one or more association values;
(c) determining one or more association signatures of the test sample based upon the association values; and
(d) comparing the association signature of the sample to an association signature of a control sample.

26. The method of claim 25, wherein the disorder is a psychotic disorder.

27. A method of prognosing a clinical outcome of a subject comprising:
(a) contacting a test sample to the array of claim 1;
(b) quantifying one or more association values;
(c) determining one or more association signatures of the test sample based upon the association values; and
(d) correlating the association signature to an association signature of a sample associated with a known clinical outcome.

28. A method of isolating a substance or molecule comprising: contacting a sample to the array of claim 1.

29. A method of detecting the presence, absence or quantity of a substance or molecule in a sample comprising:
(a) contacting the sample to an array of claim 1;
(b) quantifying one or more association values;
(c) determining one or more association signatures of the cell sample based upon the association values; and
(d) comparing the association signature of the sample to an association signature of a control sample.

30. The method of claim 29, wherein the sample is a food sample or water sample comprising substance, nucleic acid or protein, the presence of which indicates the presence of a contaminant in the food or water sample.

31. The method of claim 29, wherein the substance is a pathogen or component of a pathogen.


SEQUENCE LISTING (10 sequences)

SEQ ID NO: 1 - protein, 159 aa, Tobamovirus Ob
MPYTINSPSQFVYLSSAYADPVQLINLCTNALGNQFQTQQARTTVQQQFADAWKPVPSMTVRFPASDFYVYRYNSTLDPLITALLNSFDTRNRIIEVDNQPAPNTTEIVNATQRVDDATVAIRASINNLANELVRGTGMFNQAGFETASGLVWTTTPAT

SEQ ID NO: 2 - protein, 176 aa, Artificial Sequence
  Furovirus capsid analog
MAVKSGYTVFNKELNVMANTHAYIRLSALLSQVETWQSTRTSVLSHLGIMLNGVSKLGERNFFSRSKRFGTHTFDGDEIFCDLGGEGVSQVLTRLIVALGAAKGEGAQSRNAKRGAPPAAGQIETEEQGQTDQSLAISNALGELMTYVSSKEYTMNECYTQDSFEAKYNLKWEGSS

SEQ ID NO: 3 - protein, 198 aa, Artificial Sequence
  Hordeivrius capsid analog
MPNVSLTAKGGGHYIEDQWDTQVVEAGVFDDWWVHVEAWNKFLDNLRGINFSVASSRSQVAEYLAALDRDLPADVDRRFAGARGQIGSPNYLPAPKFFRLDKRTIAELTRLSRLTDQPHNNRDIELNRAKRATTNPSPPAQAPSENLTLRDVQPLKDSALHYQYVLIDLQSARLPVYTRKTFERELALEWIIPDAEEA

SEQ ID NO: 4 - protein, 220 aa, Artificial Sequence
  Pecluvirus capsid analog
MANISEVRRGGGHYSIASWRNHVIKQNLNHDWWIRSDRWAQLLADLRAVNYEVNSSRSEVANIINRVPKDLPADPSARFPGVVGTPGETNYSLVYYVRVEAPVREKFLGIIAAADQGKSRDVEVGRPSAPSVASGAGNQAIVPVRGVNAIRDQQPLRDGSLSFRYKLVDVELASVTQFDQVLFEETFQITWIEKDASTKTTPTASTNTSPTGVAPGDPSN

SEQ ID NO: 5 - protein, 178 aa, Artificial Sequence
  Pomovirus capsid analog
MTAAMEPHYAVFSNKMAKYAAAHPFIKYNELSETVKSWMQTRTSVMEHVNFVLGSAANLGTRGFFSRNVRFGMTNVNGDNLYADLGYLPFQNLLNALTIVLGAVGGRGKLRRNPKGESSKAAATEQINGGSDGQLNIAHCIMDINQVMSDPTILQNAVYSQSTFEEAHGLAWVYKPTA

SEQ ID NO: 6 - protein, 223 aa, Artificial Sequence
  Tobravirus capsid analog
MAMYDDEFDTKASDLTFSPWVEVENWKDVTTRLRAIKFALQADRDKIPGVLSDLKTNCPYSAFKRFPDKSLYSVLSKEAVIAVAQIQSASGFKRRADEKNAVSGLVSVTPTQISQSASSSAATPVGLATVKPPRESDSAFQEDTFSYAKFDDASTAFHKALAYLEGLSLRPTYRRKFEKDMNVKWGGSGSAPSGAPAGGSSGSAPPTSGSSGSGAAPTPPPNP

SEQ ID NO: 7 - protein, 73 aa, Artificial Sequence
  Inoviridae capsid analog
MKKSLVLKASVAVATLVPMLSFAAEGDDPAKAAFNSLQASATEYIGYAWAMVVVIVGATIGIKLFKKFTSKAS

SEQ ID NO: 8 - DNA, 30 bp, Artificial Sequence
  DNA primer 1
tacattcacttgaaccctgcatggtgcttc

SEQ ID NO: 9 - DNA, 30 bp, Artificial sequence
  SNP8NRG243177 polymorphism DNA target
gaagcaccatgcagggttcaagtgaatgta

SEQ ID NO: 10 - DNA, 30 bp, Artificial Sequence
  Non-complementray ssDNA form polymorphism
cttcgtggtacgtcccaagttcacttacat